(19) United States
(12) Patent Application Publication
(10) Pub. No.: US 2025/0283100 A1
(43) Pub. Date: Sep. 11, 2025

(54) REDUCED GLIADIN WHEAT EVENT T258

(71) Applicant: CONSEJO SUPERIOR DE INVESTIGACIONES CIENTÍFICAS (CSIC) -DELEGACIÓN ANDALUCÍA, Cordoba (ES)

(72) Inventors: María José Giménez ALVEAR, Cordoba (ES); Francisco Barro LOSADA, Cordoba (ES)

(21) Appl. No.: 18/289,615

(22) PCT Filed: May 10, 2022
(86) PCT No.: PCT/EP2022/062704

§ 371 Date: Nov. 6, 2023

(30) Foreign Application Priority Data
May 10, 2021 (EP) 21382421.2

(51) Int. Cl. C12N 15/82

(52) U.S. Cl. CPC C12N 15/8218; C12N 15/8234; C12N 15/8242

(57) ABSTRACT

The present invention relates to a transgenic wheat event, which we have designated T258, that leads to reduced gliadin content, as well as cells, seeds and plants that comprise the event. The invention also relates to methods for detecting the presence of the T258 event, probes and primers for use in the method and methods of breeding with T258 to produce wheat with reduced gliadin content.

### FIELD OF THE INVENTION

The present invention relates to a transgenic wheat event, which we have designated T258, that leads to reduced gliadin and gluten content, as well as DNA, plant cells, tissues, seeds and food compositions derived from the wheat event T258.

The invention also relates to methods for detecting the presence of the T258 event, probes and primers for use in the method, and methods of breeding with T258 to produce wheat with reduced gliadin content.

### BACKGROUND OF THE INVENTION

Cereal grains contain about 10-15% dry weight of protein, of which gluten is the most important fraction as it is the major determinant of the technological properties of baking cereals. Gluten is however, not a single protein but a complex mix of proteins that are deposited in the starchy endosperm during grain development. Gluten proteins are divided into two major fractions: the gliadins and the glutenins, which are different in terms of structure and functionality. In turn, gliadins are formed by three different fractions/types: ω-, γ-, and α gliadins. (The wheat α gliadins are sometimes also referred to as α/β gliadins based on their separation by acid electrophoresis. However, both a and β gliadins have a very similar primary structure and for these reasons are currently considered a single gliadin type (α/β type). Therefore, the terms α-gliadins and α/β-gliadins are interchangeable). The glutenins comprise two fractions; the high molecular weight (HMW) and the low molecular weight (LMW) subunits. The gliadins are generally present as monomers and contribute extensibility to wheat flour dough. The glutenins contribute elasticity to dough and form large polymers linked by disulphide bonds.

These proteins make up a complex mixture that in a typical bread wheat cultivar may be comprised of up to 45 different gliadins, 7 to 16 LMW glutenin subunits, and 3 to 6 HMW glutenin subunits. Gliadins and glutenins are not present at the same amount in the grain of cereals, and their proportions can vary within a broad range depending on both genotype (variety) and growing conditions (soil, climate, fertilisation, etc.). The ratio of gliadins to glutenins was examined in a range of cereals (Wieser and Koehler, 2009), and hexaploid common wheat showed the lowest ratio (1.5-3.1), followed by durum wheat (3.1-3.4), emmer wheat (3.5-7.6) and einkorn wheat (4.0-13.9).

In addition to their unique viscoelastic properties, gluten proteins are responsible for triggering certain pathologies in susceptible individuals: i) coeliac disease (CD), which affects both children and adults throughout the world at various frequencies (from 0.1% to >2.8%) and ii) non-coeliac gluten sensitivity (NCGS), a newly-recognised pathology of intolerance to gluten (Sapone et al., 2011) with an estimated prevalence of 6% for the USA population. At present the only treatment available for these pathologies is a complete gluten-free diet for life.

However, gliadins and glutenins do not contribute equally to CD, and gliadins are indubitably the main toxic component of gluten since most (DQ2 or DQ8)-specific CD4+T lymphocytes obtained from small intestinal biopsies from coeliac patients seem to recognize this fraction (Arentz Hansen et al., 2002). In the immune epitope database (IEDB) (http://www.iedb.org/) 190 T-lymphocyte stimulating epitopes related to CD can be found. Of these, 180 (95%) map to gliadins while only 10 (5%) map to glutenins.

However, not all gliadin epitopes are equally important in triggering CD. The α-gliadin family contain the 33-mer peptide, present in the N-terminal repetitive region, with six overlapping copies of three different DQ2-restricted T-cell epitopes with high stimulatory properties and highly resistant to human intestinal proteases (Shan et al., 2002; Tye-Din et al., 2010). The α-gliadins also contain the peptide p31-43, which has been reported to induce mucosal damage via a non-T-cell-dependent pathway (innate response) (Maiuri et al., 2003; Di Sabatino and Corazza, 2009). Moreover, an additional DQ2-restricted epitope (DQ2.5-glia-3) which partially overlaps with 33-mer peptide (Vader et al., 2002) is present in α-2-gliadins. A DQ8-restricted epitope (DQ8-glia-α1) located on the C-terminal region of α-gliadin is also present in the α-gliadins (Van de Wal Y et al., 1998).

Tye-Din et al. (Tye-Din et al., 2010) comprehensively assessed the potentially toxic peptides contained within wheat, but also barley, and rye, and identified which ones stimulate T-cells from coeliac disease patients. They found that the 33-mer peptide from wheat α-gliadin was highly stimulatory, and another peptide (QPFPQPEQPFPW, SEQ ID NO: 31) from ω-gliadin/C-hordein was immunodominant after eating wheat, barley and rye. These two peptides present in wheat, plus another from barley, can elicit 90% of the immunogenic response induced by wheat, barley and rye (Tye-Din et al., 2010).

These findings showed that the immunotoxicity of gluten could be reduced to three highly immunogenic peptides, which make the development of varieties with low-toxic epitopes more feasible.

We previously described a polynucleotide whose transcription into RNA lead to the generation of siRNA molecules that cause post-transcriptional silencing of all mRNAs coding for wheat gliadins. In particular, in WO 2010/089437 and Gil-Humanes et al. (2010) (both of which are incorporated herein by reference) we described the two vectors—pGhp-ω/α/β/γ and pDhp-ω/α/β/γ comprising ω/α/β and γ silencing sequences under the control of the wheat γ gliadin and D-hordein promoter respectively. Transformation of wheat with either of these vectors led to downregulation of ω, α/β and γ gliadins and strongly reduced expression of CD-related gliadin T-cell epitopes (Gil-Humanes et al. (2010)).

However, when any plant is transformed with foreign DNA or a transgene, the transgene is integrated at a random site in the host genome. This can result in alteration of the transgene sequence or alteration of the host genome. In particular, if the transgene is inserted into a known endogenous gene this could have a deleterious effect on another aspect of the plant's phenotype and consequently the agronomic value of the transgenic plant. The site of integration of a transgene is known as a “transgenic event”. To generate an agronomically-successful crop it is common for hundreds to thousands of transgenic events to be created, each of which are screened across seasons and locations to determine a single event with the desired phenotype, as well as optimal expression of the desired transgene both spatially and temporally.

The identification of a transgenic event that has the desired levels and patterns of transgene expression can also be used to introgress that transgene into other genetic backgrounds using conventional breeding techniques (i.e. sexual crosses). This means that the identification of such an event would allow the transgene to be introgressed into elite wheat varieties.

### SUMMARY OF THE INVENTION

An aspect of the invention relates to a nucleic acid construct consisting of a nucleotide sequence of SEQ ID NO: 1 or a variant thereof. Also provided is an expression vector comprising or consisting of the nucleic acid construct; or an isolated cell transfected with the nucleic acid construct or the expression vector.

The invention further provides a genetically modified plant wherein said plant comprises the transfected cell described herein. Preferably the nucleotide sequence is incorporated into the genome in a stable form. Preferably the plant belongs to the genus Triticum, and more preferably the plant is Triticum aestivum or Triticum turgidum. In some embodiments the plant is a Bobwhite cultivar.

Also provided is a seed derived from the genetically modified plant, wherein the seed comprises the nucleotide sequence defined in SEQ ID NO: 1 or a variant thereof. Further provided are the pollen, propagule, progeny or part of the plant derived from the genetically modified plant, wherein the pollen, propagule, progeny or part comprise the nucleotide sequence defined in SEQ ID NO: 1 or a variant thereof.

The invention further provides a wheat plant, plant part or progeny thereof comprising the genotype of the wheat event T258, wherein a representative sample of seed of said wheat event has been deposited with NCIMB under accession no. NCIMB 43626. Also provided is a seed derived from the wheat plant or derived from the progeny of the wheat plant, wherein preferably the seed comprises SEQ ID NO: 1 or a variant thereof. The invention further provides a wheat plant, plant thereof or progeny grown from the seed.

The invention also provides a seed comprising the genotype of the wheat event T258, wherein a representative sample of seed of said wheat event has been deposited with NCIMB under accession no. NCIMB 43626; as well as providing a genetically altered wheat plant or part thereof grown from the seed, or a genetically altered seed produced from the wheat plant, wherein the seed comprises the wheat event T258.

Also provided is the use of the nucleic acid construct, the expression vector or cell described herein for silencing alpha, beta, gamma and omega gliadins of Triticum spp.

The invention also provides for the use of the seed described herein in the preparation of a flour, a food composition, a vitamin or a nutritional supplement, as well as a food composition prepared from the seed described herein.

Yet further provided is a method for obtaining a genetically modified plant, comprising the following:
(a) selecting a part of the plant;
(b) transfecting at least one cell or the part of the plant of paragraph (a) with the nucleic acid construct of SEQ ID NO: 1;
(c) regenerating at least one plant derived from the transfected cell or cells;
(d) selecting one or more plants obtained according to paragraph (c) that show silencing or reduced expression and/or content of at least one of alpha, beta, gamma, and omega gliadins, reduced total gliadin content, reduced gluten content, reduced immunoreactivity and/or increased expression and/or content of glutenins.

Also provided is a method of reducing the expression and/or content of at least one of alpha, beta, gamma, and omega gliadins, reducing total gliadin content, reducing gluten content, reducing immunoreactivity and/or increasing expression and/or content of glutenins, the method comprising introducing and expressing in a plant the nucleic acid construct of SEQ ID NO: 1. Also provided is a method for producing a food composition with reduced expression and/or content of at least one of alpha, beta, gamma, and omega gliadins, reduced total gliadin content, reduced gluten content, reduced immunoreactivity and/or increased expression and/or content of glutenins, the method comprising producing a plant or part thereof in which alpha, beta, gamma, and omega gliadins are silenced as described herein and preparing a food composition from said seeds.

The invention provides a method for modulating an immune response to gliadins and/or gluten, the method comprising providing a diet of a food composition produced as described herein.

The invention further provides a wheat plant having at least one chromosome having a transgene/genomic junction polypeptide of SEQ ID NO: 28 or SEQ ID NO: 29.

Further provided is a method of producing a wheat plant with reduced expression and/or content of at least one of alpha, beta, gamma, and omega gliadins, reduced total gliadin content, reduced gluten content, reduced immunoreactivity and/or increased expression and/or content of glutenins, the method comprising:
(a) sexually crossing a first parent wheat plant, where the first parent wheat plant comprises the wheat event T258 with the genotype deposited at the NCIMB under accession no. NCIMB 43626, with a second wheat plant, whereby the second wheat plant does not comprise the wheat event T258, to produce a first generation of progeny plants;
(b) selecting plants from the first progeny with reduced gliadin and/or gluten content;
(c) selfing the first generation of progeny plants to produce a second generation of progeny plants; and
(d) selecting the plants from the second-generation progeny plants, plants with reduced gliadin and/or gluten content as described herein.

The method may further comprise backcrossing the second generation progeny plants with the second parent to produce a plurality of backcross progeny plants.

### DESCRIPTION OF THE FIGURES

The invention is further illustrated in the following non-limiting figures.

FIG. 1 shows the performance of T258 data relative to the wild control BW208. Ratios of every variable have been calculated for each experiment. A y-axis value of 1.0 indicates an equal behaviour of both transgenic and wild type lines.

FIG. 2 shows the grain moisture content (%), grain protein (% dry weight basis), grain starch (% dwb) and kernel weight in grams (KW) of lines BW208 and T258.

FIG. 3 shows box plots of grain prolamin contents of lines BW208 and T258 as quantified by RP-HPLC. Prolamins are endosperm storage proteins (gluten proteins). Gliadin and Glutenin are monomeric and polymeric prolamin fractions, respectively. Omega, Alpha and Gamma are gliadin fractions. High molecular weight (HMW) and Low Molecular Weight (LMW) are glutenin fractions. Gliadin proteins (particularly alpha-gliadins) hold most of the immunogenic epitopes toxic for celiac patients.

FIG. 4 shows a comparison of the average two-year gluten content (ppm) of T258 and BW208 as measured by moAb R5.

FIG. 5 shows alveograms of doughs of lines T258 (A) and BW208 (B) harvested in June 2019. P: tenacity; L: extensibility; and W: dough strength, respectively. The Chopin Alveograph is a test commonly used to help determine the baking potential of wheat by measuring its characteristics.

FIG. 6A Melting curves*A) for T7 generation of T258-2 dilutions and B) the wild type BW208 of OA-sense_i (red), TaFAd (blue) and pinb (green) amplification products.

FIG. 7. Standard curves produced by the CFX thermocycler software: ECFX efficiencies, slopes and determination coefficient (R2) for A) OA, B) TaFAd, and C) pinb primer pairs, respectively.

FIG. 8. Amplification Curves of the target and the reference genes for A) the T7 generation of T258-2 dilutions and B) the wild type BW208: OA-sense_i (red), TaFAd (blue) and pinb (green) for the. The wild type yields an unspecific amplification product.

FIG. 9. DNA Agarose Electrophoresis for sample quality check. (A) 1-3 lanes correspond to 100 ng of the corresponding DNA. (B) Dral enzyme digestion

FIG. 10. DNA digestions with four different standard frequently-cutting restriction enzymes.

FIG. 11. Genome Walker Adaptor provided with the Universal GenomeWalker 2.0 kit (Takara Bio USA, Inc.). An amine group is blocking the exposed 3′ end of the adaptor to prevent extension of the 3′ end (which would create an AP1 binding site).

FIG. 12. Schematic representation of the transgene. Restriction enzymes cutting site are indicated in dark blue, insertion-specific primers and their amplification direction are indicated as green arrows. Numbers in grey represent DNA size in base pairs (bp).

FIG. 13. Primary PCR for Genome Walking

FIG. 14. Nested or Secondary PCR for Genome Walking

FIG. 15. Representation of the insertion site; aligning of the Triticum aestivum Paragon Elv1.1 sequence obtained by blast with the two flanking sequences from GW products (annotated as GW). Primer from table 14 are represented above the aligning

FIG. 16. Detection of transgene-genomic insert junctions or intact insertion site by PCR.

FIG. 17. Alignment of the Triticum aestivum Paragon Elv1.1 scaffold 012658, the corresponding intact sequence of the insertion site in BW208 wildtype and the T258 flanking consensus sequences. Regions in T258 that correspond with the transgene are indicated by arrows

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene”, “allele” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. Thus, according to the various aspects of the invention, genomic DNA, cDNA or coding DNA may be used. In one embodiment, the nucleic acid is cDNA or coding DNA.

The term ‘variant’ refers to a nucleotide sequence where the nucleotides are substantially identical to the described non-variant sequence. The variant may be achieved by modifications such as insertion, substitution or deletion of one or more nucleotides. In a preferred embodiment, the variant has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to any one of the sequences described herein, preferably over the full length of the sequence. In one embodiment, sequence identity is at least 90%. Sequence identity can be determined by any one known sequence alignment program in the art.

In a first aspect of the invention, there is provided a nucleic acid construct consisting of a nucleotide sequence of SEQ ID NO: 1 or a variant thereof. In a further aspect of the invention, there is provided an expression vector comprising or consisting of the nucleic acid construct.

The inventors have shown that it is possible to reduce the expression and/or content of at least one of alpha, beta, gamma, and omega gliadins, reduce total gliadin content, reduce gluten content, reduce immunoreactivity (to gliadins/gluten) and/or increase expression and/or content of glutenins by introducing and expressing in a plant or plant part thereof a nucleic acid construct consisting only of the nucleotide sequence defined in SEQ ID NO: 1. As shown in SEQ ID NO: 1 this nucleotide sequence comprises or consists in the 5′ to 3′ direction of transcription:
a gamma-gliadin promoter (bold);
a sense RNAi sequence for omega-gliadin and a sense RNAi sequence for alpha/beta and gamma gliadins (dashed line), flanked by gateway recombination sites (underlined);
a Ubi1 intron from Z. mays;
a anti-sense RNAi sequence for alpha/beta and gamma gliadins (dashed line) flanked by gateway recombination sites (underlined); and
a NOS terminator sequence (double underlined).

As this sequence contains no other genes (such as, for example, genes for herbicide and antibiotic resistance, which are commonly found in constructs for bacterial transformation), the presently described “back-bone” free nucleic acid construct complies with regulatory provisions around introduction of unnecessary genetic matter.

In another aspect of the invention, there is provided an isolated cell transfected with the nucleic acid construct described herein or the expression vector described herein. Preferably the cell is a plant cell, more preferably a wheat cell.

In a further aspect of the invention, there is provided a genetically modified plant wherein said plant comprises the transfected cell described herein. In a preferred embodiment, the nucleotide sequence is incorporated in the plant genome in a stable form. As used herein, “stable” means that the nucleotide sequence is integrated into the plant genome and continues to be integrated in the genome of progeny cells

The term “introduction”, “transfection” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The resulting transformed plant cell may then be used to regenerate a transformed plant in a manner known to persons skilled in the art.

The transfer of foreign genes into the genome of a plant is called transformation.

Transformation of plants is now a routine technique in many species. Any of several transformation methods known to the skilled person may be used to introduce the nucleic acid construct into a suitable ancestor cell. The methods described for the transformation and regeneration of plants from plant tissues or plant cells may be utilized for transient or for stable transformation. As used throughout “stable” means that the gene or nucleic acid sequence integrated into the plant genome and continues to be integrated in the genome of progeny cells

Transformation methods include the use of liposomes, electroporation, chemicals that increase free DNA uptake, injection of the DNA directly into the plant (microinjection), gene guns (or biolistic particle delivery systems (biolistics), lipofection, transformation using viruses or pollen and microprojection. Methods may be selected from the calcium/polyethylene glycol method for protoplasts, ultrasound-mediated gene transfection, optical or laser transfection, transfection using silicon carbide fibers, electroporation of protoplasts, microinjection into plant material, DNA or RNA-coated particle bombardment, infection with (non-integrative) viruses and the like. Transgenic plants can also be produced via Agrobacterium tumefaciens mediated transformation, including but not limited to using the floral dip/Agrobacterium vacuum infiltration method as described in Clough & Bent (1998) and incorporated herein by reference.

Accordingly, in one embodiment, at least one nucleic acid construct as described herein can be introduced into at least one plant cell using any of the above described methods.

Following DNA transfer and regeneration, putatively transformed plants may also be evaluated, for instance using PCR to detect the presence of the gene of interest, copy number and/or genomic organisation. Alternatively or additionally, integration and expression levels of the newly introduced DNA may be monitored using Southern, Northern and/or Western analysis, each technique being well known to persons having ordinary skill in the art.

The generated transformed plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques.

In a further related aspect of the invention, there is also provided, a method of obtaining a genetically altered plant as described herein, the method comprising
a. selecting a part of the plant;
b. transfecting at least one cell of the part of the plant of paragraph (a) with at least one nucleic acid construct as described herein using the transfection or transformation techniques described above;
c. regenerating at least one plant derived from the transfected cell or cells;
d. selecting one or more plants obtained according to paragraph (c) that show silencing or reduced expression and/or content of at least one of alpha-, gamma- and/or omega gliadins, reduced total gliadin content, reduced gluten content, a reduced gliadin to glutenin ratio and/or increased expression and/or content of glutenins.

In another embodiment, the method may further comprise the step of screening the genetically altered plant for the presence of exogenous nucleic acid, i.e. the nucleotide sequence of SEQ ID NO: 1, wherein the method also comprises obtaining a DNA sample from a transformed plant and carrying out DNA amplification to detect the presence of the exogenous DNA. As an example, primers that can be used for such DNA amplification are described in SEQ ID NOs 2 and 3.

In a further embodiment, the methods comprise generating stable T2 plants that comprise SEQ ID NO: 1 stably incorporated into the genome, and preferably into the genome of the seeds. A plant obtained or obtainable by the methods described above is also within the scope of the invention.

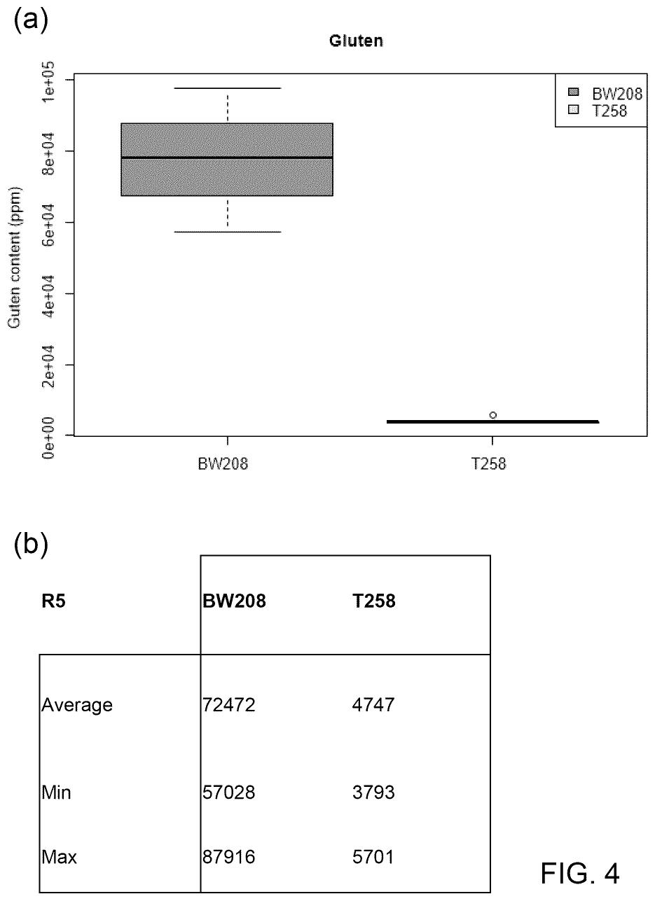

In a preferred aspect of the invention, the invention relates to a mutant wheat genotype (Triticum aestivum), designated Accession Number NCIMB 43626 deposited on 16 Jun. 2020 under the Budapest Treaty at NCIMB (National Collection of Industrial and Marine Bacteria) Ltd, Ferguson Building, Craibstone Estate, Bucksburn, Aberdeen, AB21 9YA, Scotland by the Institute of Sustainable Agriculture CSIC (Consejo Superior De Investigaciones Cientificas), Alameda del Obispo S/N, 14004 Cordoba, Spain. The depositor's reference is Triticum aestivum T258. The deposited material was found viable in a test performed on 16 Jun. 2020. The invention thus relates to any wheat plants, parts thereof, including seeds, having this genotype. This mutant is described herein as T258. All references to “T258” in this application are to this deposit. The T258 “event” was produced by transformation of the BW208 wheat line with the above-described nucleic acid construct (consisting of the nucleotide sequence of SEQ ID NO: 1). As described in the examples below, plants of the T258 line have significantly reduced levels of at least one of alpha, beta, gamma, and omega gliadins, reduced total gliadin content, significantly reduced gluten content (and consequently reduced immunoreactivity as shown by reactivity with the R5 antibody in FIG. 4) and increased expression/content of glutenins. This wheat line that is low-gliadin and therefore, low-gluten can be used to produce low gluten foodstuffs. The line can also serve as source material in plant breeding programs to introgress this trait into elite wheat varieties.

As used herein, an “event” refers to a transgenic event. An event is produced by transformation of plant cells with heterologous nucleic acid (also referred to as a transgene), such as the nucleotide sequence of SEQ ID NO: 1. The event also refers to the regeneration of a population of plants from the insertion of the transgene into the genome of the plant, and the selection of a plant that contains insertion of the transgene at a particular genomic location. The term “event” as used herein refers to the original transformant and the progeny from the original transformant that carry the transgene.

The term “event” also refers to progeny obtained from a sexual cross between a first parent carrying the transgene and a second parent (without the transgene) or equally, to progeny obtained from the sexual cross of one parental line (i.e. selfing). Even after backcrossing to the second, non-transformant parent, the transgene from the first parent will be present in the progeny at the same chromosomal location.

Accordingly, in a further aspect of the invention, there is provided a wheat plant, plant part or progeny thereof comprising the genotype of the wheat event T258, wherein a representative sample of seed of said wheat event has been deposited with NCIMB under accession no. NCIMB 43626.

In a further aspect of the invention, there is provided a seed comprising the wheat event T258, wherein the seed comprises SEQ ID NO: 1, and wherein furthermore, a representative sample of wheat event T258 has been deposited with the NCIMB under accession no. NCIMB 43626. In a further aspect, there is also provided a genetically altered plant or plant part thereof grown from these seeds, and seeds obtained from these plants, where the seed comprises the wheat event T258 as well as subsequent genetically altered plants or parts thereof grown from these seeds.

In another aspect of the invention, there is provided a method of producing a wheat plant with low (reduced) gliadin and/or gluten as described herein, the method comprising (a) sexually crossing a first parent wheat plant, where the first parent wheat plant comprises the wheat event T258 with the genotype deposited at the NCIMB under accession no. NCIMB 43626, with a second wheat plant, whereby the second wheat plant does not comprise the wheat event T258, to produce a first generation of progeny plants; (b) selecting plants from the first progeny with reduced gliadin and/or gluten content; (c) selfing the first generation of progeny plants to produce a second generation of progeny plants and (d) selecting from the second generation progeny plants, plants with reduced gliadin and/or gluten content as described herein. In a further embodiment, the method may further comprise the step of backcrossing the second generation progeny plants with the second parent (which does not comprise the wheat event T258) to produce a plurality of backcross progeny plants. Preferably, the backcross progeny comprise SEQ ID NO: 1, integrated into the genome (at a detectable level). Hybrid seed obtained or obtainable from this method are also part of the invention.

In an alternative embodiment, the T258 event plants may be self-pollinated to produce inbred lines that are homozygous for the T258 event.

A genetically altered plant of the present invention may also be obtained by transference of SEQ ID NO: 1 by crossing, e.g., using pollen of the genetically altered plant described herein to pollinate a wild-type or control plant, or pollinating the gynoecia of the genetically altered plants described herein with other pollen that does not contain SEQ ID NO: 1 or a variant thereof incorporated into its genome. The methods for obtaining the plant of the invention are not exclusively limited to those described in this paragraph;

for example, genetic transformation of germ cells from the ear of wheat could be carried out as mentioned, but without having to regenerate a plant afterward.

In another embodiment, the present invention provides regenerable mutant plant cells for use in tissue culture. The tissue culture will preferably be capable of regenerating plants having essentially all of the physiological and morphological characteristics of the foregoing genetically altered wheat plant, and of regenerating plants having substantially the same genotype. Preferably, the regenerable cells in such tissue cultures will be callus, protoplasts, meristematic cells, cotyledons, hypocotyl, leaves, pollen, embryos, roots, root tips, anthers, pistils, shoots, stems, petiole, flowers, and seeds. Still further, the present invention provides wheat plants regenerated from the tissue cultures of the invention.

In another aspect of the invention, there is provided a method of reducing the expression and/or content of at least one of alpha, beta, gamma, and omega gliadins, reducing total gliadin content, reducing gluten content, reducing immunoreactivity and/or increasing expression and/or content of glutenins, the method comprising introducing and expressing in a plant the nucleic acid construct described herein.

In a further aspect of the present invention there is provided the use of a nucleic acid construct as defined herein to silence or reduce the expression and/or content of at least one of alpha-, gamma- and/or omega gliadins of the Triticum spp.

As used herein a reduction in reference to any of alpha-, gamma- and/or omega gliadin expression and/or content levels, total gliadin, gluten content and/or gluten immunoreactivity is meant a reduction of at least 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% compared to the levels in a control or wild-type plant. A 100% reduction can also be considered as silencing. Alternatively, said reduction is at least a two, three, four, five, six, seven, eight, nine or ten-fold or up to a twenty-fold reduction compared to the levels in a control or wild-type plant.

These reductions can be measured by any standard technique known to the skilled person. For example, a reduction in the expression and/or content levels of at least one of alpha-, gamma- and/or omega gliadin and total gliadin levels may be a measure of protein and/or nucleic acid levels and can be measured by any technique known to the skilled person, such as, but not limited to, any form of gel electrophoresis or chromatography (e.g. HPLC) as described in the examples. Techniques for measuring total gluten and gluten immunoreactivity are also known to the skilled person, but again as a non-limiting example, can include measurement using the monoclonal antibodies R5 and G12, as described in the examples.

The methods described herein may also further comprise the steps of measuring (as described above) and/or selecting plants with reduced alpha-, gamma- and/or omega gliadin expression and/or content levels, total gliadin, gluten content and/or gluten immunoreactivity. The method may further comprise the step of regenerating a selected plant as described herein.

In a further aspect of the invention, there is provided a method for producing a food composition with reduced expression and/or content of at least one of alpha, beta, gamma, and omega gliadins, reduced total gliadin content, reduced gluten content, reduced immunoreactivity and/or increased expression and/or content of glutenins, the method comprising producing a plant or part thereof in which alpha, beta, gamma, and omega gliadins are silenced as described above and preparing a food composition from said seeds. In a preferred aspect of the invention, there is provided a method of producing a food composition from the seeds derived from the T258 event plants, as described above.

In a further aspect of the invention, there is provided a method for modulating an immune response to gliadins and/or gluten, the method comprising providing a diet of a food composition produced as described above.

In another aspect of the invention, there is provided the use of a seed derived from a genetically altered plant as described herein, in the preparation of a food composition. Preferably, the seed is derived from a plant of the T258 line as described above. The food composition is prepared from, but not limited to, the flour and/or semolina of the seeds of the invention, combined or not with other flours and/or semolinas, or other compounds.

The term “flour” as it is understood in the present invention refers to the product obtained by milling of any seed or plants of the genus Triticum, with the bran or husk of the seed removed to a greater or lesser degree.

The term “semolina” refers to coarse flour (slightly milled wheat seeds), i.e., fragments of the endosperm with a variable amount of seed husks.

The prepared food is selected from, but not limited to, the list comprising bread, bakery products, pastries, confectionery products, food pasta, food dough, grains, drinks, or dairy products.

Another aspect of the invention is use of the composition of the invention to prepare a food product, vitamin supplement, or nutritional supplement. As understood in the present invention, a food product fulfils a specific function, such as improving the diet of those who consume it. For this purpose, a vitamin and/or nutritional supplement may be added to the food product.

The food product that comprises the food composition of the present invention may be consumed even by persons who are allergic to gluten, i.e., suffer from celiac disease.

In a further aspect of the invention, there is provided a method for producing a food composition, wherein said food composition preferably has a reduced gliadin and/or gluten content and/or reduced immunotoxicity, the method comprising producing a genetically altered plant, characterised in that said plant has reduced expression and/or content of at least one of alpha-, gamma- and/or omega gliadins, reduced total gliadin content, reduced gluten content, a reduced gliadin to glutenin ratio and/or increased expression and/or content of glutenins.

Preferably, said plant is obtained by transfecting at least one plant cell with at least one nucleic acid construct as described herein in the seeds of said plant, producing seeds from said plant and preparing a food composition from said seeds. In a preferred embodiment, the plant or plant seeds are derived from plants of the T258 event, as described above.

In another aspect of the invention, there is provided a method for modulating an immune response to gliadins and/or gluten or affecting or modulating a T-cell response to gluten in a subject, the method comprising providing a diet of a food composition as described herein to a subject in need thereof.

The term “plant” as used herein encompasses whole plants, ancestors and progeny of the plants and plant parts, including seeds, fruit, shoots, stems, leaves, roots (including tubers), flowers, and tissues and organs, wherein each of the aforementioned comprise the gene/nucleic acid construct/RNA molecule of interest. As used herein, the term “progeny” includes offspring of any generation of parent plant that comprises SEQ ID NO: 1 integrated into its genome, and more specifically comprises the wheat event T258. As such, the progeny may be of any generation. The term “plant” also encompasses plant cells, suspension cultures, protoplasts, callus tissue, embryos, meristematic regions, gametophytes, sporophytes, pollen and microspores, again wherein each of the aforementioned comprises SEQ ID NO: 1.

The invention also extends to harvestable parts of a mutant plant of the invention as described above such as, but not limited to seeds, leaves, fruits, flowers, stems, roots, rhizomes, tubers and bulbs. The invention furthermore relates to products derived, preferably directly derived, from a harvestable part of such a plant, such as dry pellets or powders, oil, fat and fatty acids, flour, starch or proteins. The invention also relates to food products and food supplements comprising the plant of the invention or parts thereof.

The wheat plant is selected from the list that includes, but is not limited to, Triticum aestivum, T. aethiopicum, T. araraticum, T. boeoticum, T. carthlicum, T. compactum, T. dicoccoides, T. dicoccum, T. durum, T. ispahanicum, T. karamyschevii, T. macha, T. militinae, T. monococcum, T. polonicum, T. repens, T. spelta, T. sphaerococcum, T. timopheevii, T. turanicum, T. turgidum, T. urartu, T. vavilovii and T. zhukovskyi.

According to another embodiment the various aspects of the invention described herein, the plant is of the species Triticum aestivum. According to another preferred embodiment, the plant belongs to the cultivar Bobwhite, and more preferably the cultivar BW208 (Bobwhite). Bobwhite is the name of the cultivar obtained from the International Maize and Wheat Improvement Center (CIMMYT). BW208 is a Bobwhite line.

A control plant as used herein is a plant which has not been modified according to the methods of the invention. Accordingly, the control plant does not have SEQ ID NO: 1 incorporated into the plant genome. In one embodiment, the control plant is a wild type wheat plant. In another embodiment, the control plant is a plant that does not have SEQ ID NO: 1 incorporated into the plant genome, but is otherwise modified. The control plant is typically of the same plant species, preferably the same ecotype or the same or similar genetic background as the plant to be assessed.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification, including reference to sequence database identifiers, are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The invention is further described in the following non-limiting examples.

### Example 1

### Generation of Plants

The DNA fragment of SEQ ID NO: 1 (DNA fragment containing the Ghp_omega/alpha construction) was obtained by double digestion of the plasmid pGhp_omega/alpha (GenBank accession HM352558.1) with the restriction enzymes Haell and Bsal. The digestion products were separated by agarose electrophoresis. The larger band, containing the construct, was cut from the gel and purified using a GenElute™ Gel Extraction Kit. The total amount of fragment obtained by purification of the bands recovered from the gel was diluted until obtaining a concentration of 172 ng/μL.

The plant transformation method applied was biolistic, that is, the direct transfer of genes by microprojectile using a particle gun for immature embryo cultures according to the method published in Barro et al. (1998) with some modifications. Briefly, immature scutella of wheat (Triticum aestivum L.) line were used as target tissues for transformation by particle bombardment. Plants were grown in greenhouse under natural day/night light regime. Caryopses were harvested and explants isolated as described by Barcelo and Lazzeri (1995). For each bombardment about 35 immature scutella were placed in a 90-mm petri dish containing induction medium. Explants were precultured in darkness at 24° C. for 1 day in the induction media MP4 (MS macrosalts (Murashige and Skoog 1962), L microsalts (Lazzeri et al. 1991), MS vitamins (Murashige and Skoog 1962), FeNaEDTA (Sigma, 10 g-3·L-1), L1 amino acids (Lazzeri et al. 1991) and 30 g·L-1 sucrose, plus 4 g-3·L-1 picloram), and, prior to bombardment, were placed in the centre of a petri dish containing MP4MO media (MP4 media plus 0.4M mannitol).

The Ghp omega/alpha DNA fragment, was precipitated onto gold particles following the protocol of Barcelo and Lazzeri (1995). Bombardments were carried out at a distance of 6 cm from the stopping plate using a PDS 1000/He gun (BioRad) at a helium pressure of 1100 psi, and 3 pmol DNA/mg gold, 60 μg gold were used in each shot. Two hours after bombardment, explants were spread over the surface of the medium in the original dishes and cultured at 24° C. in darkness for 3 weeks. The bombardment of a variable number of plates was repeated four times for a sum of 737 explants.

Once putatively introduced the gene explants were in vitro cultured in a suitable medium to promote somatic embryogenesis. A total number of 542 explants yielded embryogenic calli, which were successively transferred to regeneration media until whole plants were obtained. Since the construct does not contain any genes that allow selection, each of the 1022 plants obtained in this process was individually transferred to a pot. The presence of the gene of interest was examined at this stage by analyzing the plant DNA using qPCR.

Up to 5 T1 seeds of TO plants carrying the genetic construction were analyzed by acid polyacrylamide gel electrophoresis (A-PAGE). Plant 3000, derived from embryo 22 of the sixth bombarded plate, produced only two seeds, R625 and R626, whose half grains showed a silenced profile. The embryos of these T1 seeds were sown and raised in pots in a greenhouse.

Ten T2 half seeds from each T1 plant were analyzed by A-PAGE. All the descendants of R626 analyzed showed a silenced profile. The embryos of these ten seeds (T2) were sown and raised in pots.

Twenty half grains of the next generation (T3) were analyzed. All the descendants of the T258 plant showed a silenced profile. The plant was considered homozygous for the silencing and the line was named T258.

In a new multiplication cycle, T4 seeds were obtained from the T258 line. From this generation on, all the analyzes (HPLC, R5, G12, . . . ) have been done on flour of bulked seeds, confirming the stability of the silencing.

During the following two growing seasons, T258 was multiplied again, so that the HPLC analyzes of the gliadin content have once again confirmed the stability of the character.

### Example 2

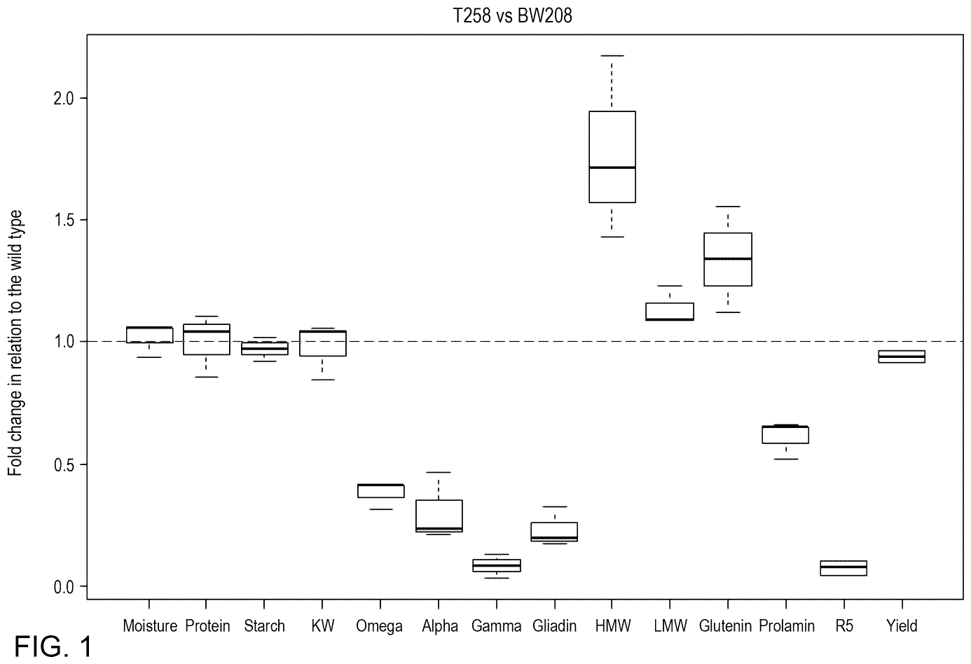

In Example 2, the performance of the lines T258 (silenced) and BW208 (wild type) was compared in three different experiments carried out in different years. Excepting year 2019, the experiments were carried out in very small plots (ten plants per plot and two reps in 2017, and five plants per elementary plot and three reps in 2018). Samples of each of T258 and BW208 were analyzed to determine the following variables: kernel weight (KW), grain moisture content (Moisture), grain protein (Protein), grain starch (Starch), gluten content by moAb R5 (R5), fructan content (Fructan), and prolamin and its fractions (quantified by HPLC). Glutenin and gliadin are polymeric and monomeric prolamin fractions, respectively. Omega, alpha and gamma are gliadin fractions. Ratios of T258 to BW208 values are presented in FIG. 1 and Table 1. Calculating ratios allows, despite the different nature of the experiments, to compare the variation of the performance of line T258 related to its control line BW208 within each of them.

TABLE 1
Values of the ratios T258 vs BW208 in 2017, 2018 and 2019. For
variables other than those related to prolamins, the ratio mean
value is about 1.00, meaning there are no differences between
T258 and its parental line. The gliadin fractions contents and
the toxic gluten content measure by the R5 moAb are clearly reduced
in T258, whereas both glutenin fractions are increased in this
line related to BW208. T258 yield is slightly decreased.

| Year | 2017 | 2018 | 2019 | Mean* |
|---|---|---|---|---|
| Moisture | 0.94 | 1.05 | 1.05 | 1.01 |
| Protein | 1.10 | 1.04 | 0.86 | 1.00 |
| Starch | 0.92 | 0.97 | 1.02 | 0.97 |
| KW | 1.05 | 1.04 | 0.84 | 0.97 |
| Omega | 0.41 | 0.41 | 0.31 | 0.38 |
| Alpha | 0.23 | 0.47 | 0.21 | 0.28 |
| Gamma | 0.08 | 0.13 | 0.03 | 0.07 |
| Gliadin | 0.19 | 0.32 | 0.17 | 0.22 |
| HMW | 2.18 | 1.72 | 1.43 | 1.75 |
| LMW | 1.23 | 1.09 | 1.08 | 1.13 |
| Glutenin | 1.55 | 1.34 | 1.12 | 1.33 |
| Prolamin | 0.66 | 0.65 | 0.52 | 0.60 |
| R5 | 0.10 | — | 0.04 | 0.07 |
| Yield | | 0.96** | 0.91 | 0.94 |

*Geometric mean
**Yield data have been extrapolated from very small plot data and should therefore be viewed with caution

Similar data is presented for year 2020. Again, all gliadin fractions are notably reduced, as well as the toxic gluten content measure by the R5 moAb. Yield is slightly increased.

TABLE 2
Mean values of agronomic and protein features
of T258 and BW208 lines for year 2020.

| | | | Ratio |
|---|---|---|---|
| | | | T258 vs |
| | BW208 | T258 | BW208) |
| Anthesis (mm/dd) | 03/11 | 03/11 | 1 |
| Yield (kg ha⁻¹)* | 4470.7 | 4520.6 | 1.01 |
| Kernel weight (g) | 50.2 | 45.2 | 0.90 |
| OMEGA (g kg−¹) | 6.0 | 3.4 | 0.57 |
| ALPHA (g kg−¹) | 20.0 | 2.6 | 0.13 |
| GAMMA (g kg−¹) | 13.6 | 0.4 | 0.03 |
| GLIADINS (g kg−¹) | 39.5 | 6.4 | 0.16 |
| HMW (g kg−¹) | 8.2 | 19.3 | 2.37 |
| LMW (g kg−¹) | 13.0 | 20.4 | 1.58 |
| GLUTENINS (g kg−¹) | 21.1 | 39.8 | 1.88 |
| PROLAMINS (g kg−¹) | 60.7 | 46.2 | 0.76 |
| GLIADINS/GLUTENINS | 1.9 | 0.2 | 0.09 |
| R5 (mg kg−¹) | 88287 | 4617 | 0.05 |
| Moisture (%) | 9.5 | 9.3 | 0.98 |
| PROTEIN (% dwb) | 10.9 | 11.1 | 1.02 |

*Yield data have been extrapolated from small plot data

Protein data were determined by RP-HPLC and expressed in g·kg⁻¹ of whole wheat flour.

### Example 3: Grain Performance and Agronomic Performance

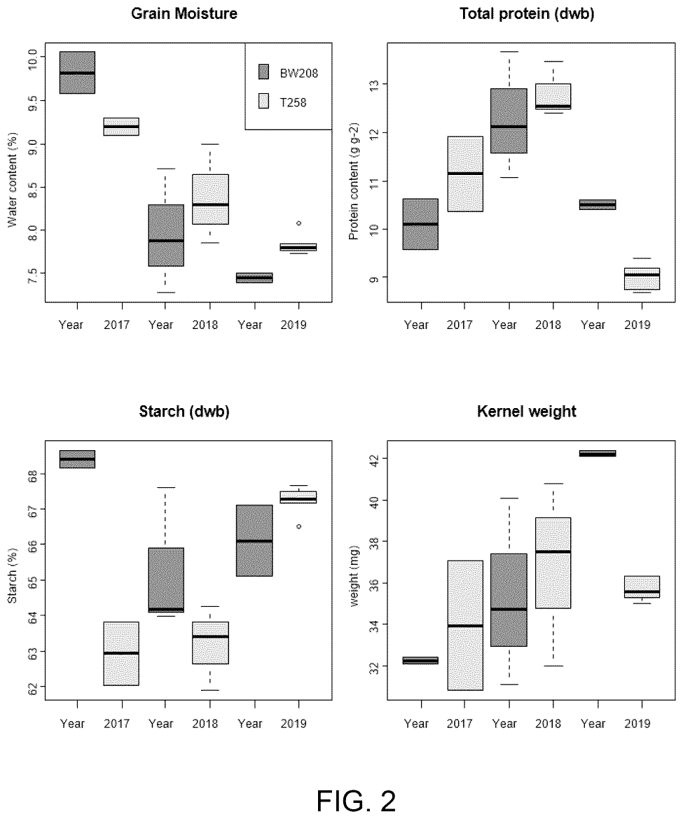

Absolute values of the above mentioned experiments are presented below. As shown in FIG. 2 and Tables 3 and 4.

TABLE 3
Mean values of: percentage of grain moisture, total
protein, and starch of the lines T258 and BW208.

| | | Water | Protein | Starch |
|---|---|---|---|---|
| Line | Year | (%) | (% dwb) | (% dwb) |
| BW208 | 2017 | 9.8 | 10.1 | 68.4 |
| T258 | 2017 | 9.2 | 11.1 | 62.9 |
| BW208 | 2018 | 8.0 | 12.3 | 65.3 |
| T258 | 2018 | 8.4 | 12.8 | 63.2 |
| BW208 | 2019 | 7.5 | 10.5 | 66.1 |
| T258 | 2019 | 7.85 | 9.01 | 67.21 |

TABLE 4
Date of anthesis and mean values of kernel weight
and yield of the lines T258 and BW208.

| | | | Kernel | |
|---|---|---|---|---|
| | | Anthesis | weight | Yield |
| Line | Year | (mm/dd)) | (g) | (kg · ha⁻¹) |
| BW208 | 2018 | 04/14 | 35.00 | 3801* |
| T258 | 2018 | 04/10 | 37.00 | 3652* |
| BW208 | 2019 | 04/01 | 42.25 | 5225 |
| T258 | 2019 | 03/28 | 35.70 | 4775 |

*Yield data have been extrapolated from small plot data.

### Example 4: Prolamin Content

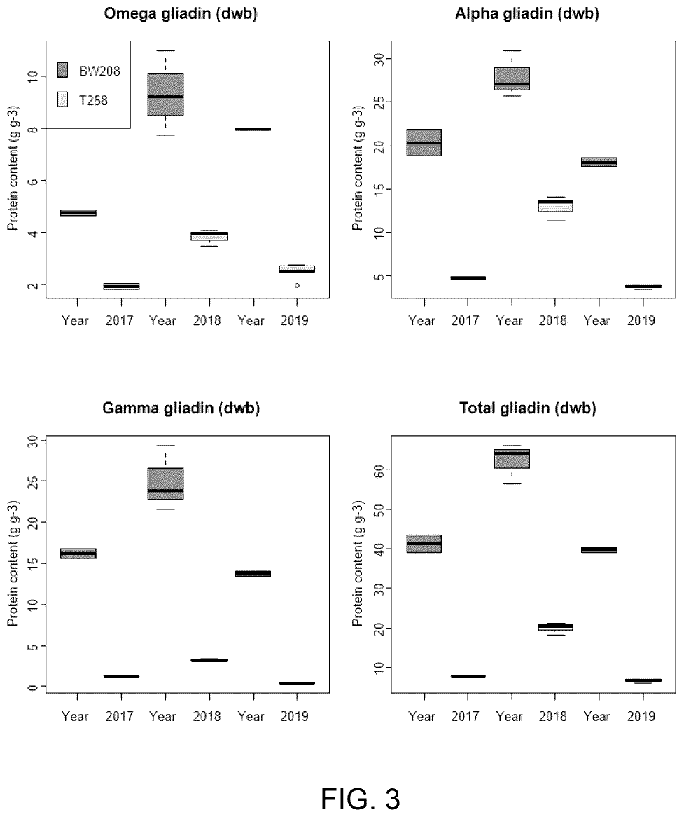

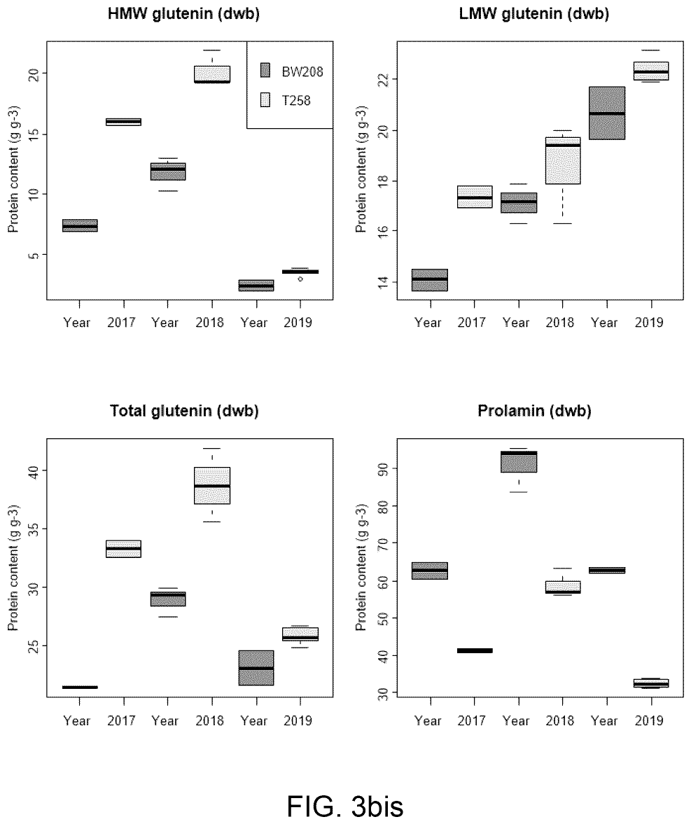

Protein content was quantified by RP-HPLC according to Wieser & Kiefer (Wieser H., Kieffer R. 2001. Correlations of the Amount of Gluten Protein Types to the Technological Properties of Wheat Flours Determined on a Micro-scale. Journal of Cereal Science, 34, 1:19-27) with minor modifications (FIG. 3 and Table 5). Every year, the amounts of each gliadin fraction of T258 were much lower than those of BW208. As also shown in Table 5, in relation to its wild type parent, the silenced line T258 shows consistent reduction in gliadin content (around 75%), partially compensated by a higher amount of HMW glutenins. The gamma gliadin is the most reduced protein (more than 90%)

TABLE 5
Comparison of the average three-year RP-HPLC values
of lines T258 and BW208. Values are expressed in g ·
kg⁻¹ of whole wheat flour.

| | |
|---|---|
| BW208 | T258 |

| Protein | Average | Rank | Average | Rank |
|---|---|---|---|---|
| Omega | 7.3 | 4.8-9.3 | 2.8 | 2.0-3.8 |
| Alpha | 22.1 | 18.1-27.9 | 7.2 | 3.8-13.0 |
| Gamma | 18.3 | 13.8-24.9 | 1.6 | 0.4-3.2 |
| Gliadin | 47.7 | 39.7-62.1 | 11.5 | 6.7-20.0 |
| HMW | 7.2 | 2.4-11.8 | 13.2 | 3.4-20.2 |
| LMW | 17.3 | 14.1-20.7 | 19.4 | 17.3-22.4 |
| Glutenin | 24.4 | 21.4-28.9 | 32.6 | 25.8-38.8 |
| Prolamin | 72.1 | 62.7-91.0 | 44.2 | 32.5-58.7 |

### Example 5: Gluten Content and Alveograph Quality Parameters

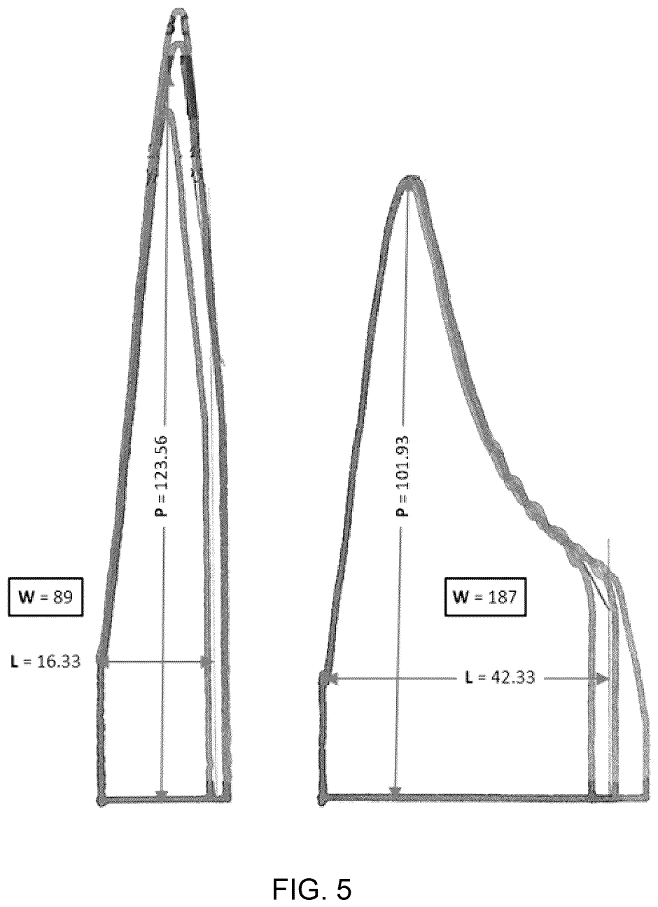

As shown in FIG. 4, the decrease in gluten content in line T258 relative to the wild type is more than 90%, even higher than the gliadins reduction. Finally, we assessed the baking potential of the T528 line. As shown in FIG. 5, breadmaking quality of T258 and BW208 are different according to Chopin's alveograph parameters: a higher P and lower L and W. This means that hydration of T258 dough should be adapted for adequate bread making.

### Example 6: Determination of Copy Number of RNAi Silencing Fragment in T258 Line

Classically, Southern blotting was the method for quantifying gene copy number in a genome. But this technique has demonstrated not to be able to accurately determine multiple copies integration of a single gene commonly found in transgenic plants. Quantitative PCR is a simple and powerful tool for the determination of transgene copy number, both by using probes or intercalating fluorophores as SYBR green. The method consists in comparing for each individual the quantity of target gene with the quantity of a gene of known copy number. For hexaploid wheat, the pinb gene, located of chromosome 5D has been proposed as a single copy reference gene (Li et al. 2004).

The number of copies of the construction inserted in T258 genome has been calculated by means of qPCR in a CFX BioRad thermocycler. Three primers pairs were designed for 1) the amplification of a fragment of the omega-alpha construction, which comprises part of the sense inverted repeat and the UBI intron (OA-sense i); 2) the amplification of the exon 6 of the three copies (personal communication) of the TaFAd gene (Probable ATP synthase 24 kDa subunit, mitochondrial [Source: Projected from Arabidopsis thaliana (AT2G21870) UniProtKB/Swiss-Prot; Acc: Q9SJ12]) present in Triticum aestivum; and 3) the amplification of the Puroindoline b gene (pinb), above mentioned.

DNA from different plants of the T258 line was extracted and subjected to qPCR: one T₂ generation from T258 line, and two T₇ generation from T258 line. The DNA of the wild type BW208 and no-template were used as control samples.

### In Silico Specificity Analysis of the Primer Pairs

According to D'Haene et al. (2010), a primer pair may be considered as specific if the following requirements are fulfilled:
An expect value close to zero.
An identities value of 100% for both the forward and reverse primer.
Primers should be located on complementary strands.
An amplicon length between 80 and 150 bp.
Primers should only match via BLAST analysis at the sequence of interest.

Prior to the qPCR analysis, a BLAST search was performed against the wheat assembly IWGSC to verify the specificity of the three primers pairs by using the web tool https://plants.ensembl.org/Triticum_aestivum/Tools/Blast adjusted for short sequences.

The conditions of the amplification, reaction volume, materials analyzed, and the polymerase and chemistry used are detailed in Table 6.A. Tables 6.B and 5.C describe the sequences of the primers and the composition of the reaction, respectively.

TABLE 6
Composition, components and conditions for quantitative PCR.
A)

| | |
|---|---|
| Thermocycler | CFX BioRad |
| Amplification protocol | Two-step protocol; Ta = 62º C.; |
| | 40 cycles |
| Volume | 20 μL |
| Samples | T258, BW208, H2O |
| Polymerase and buffer reagent | iTaq ™ Universal SYBR ® Green |

B)

| Primers for detection target gene | Sequence |
|---|---|
| OAsense_i-F (SEQ ID NO: 4) | CGGTGCCAGGCCATCCACAA |
| OAsense_i-R (SEQ ID NO: 5) | GCGGCGTACCTTGAAGCGGA |
| Primers for reference genes | |
| TaFAD6-F (SEQ ID NO: 6) | GCTTGGCATTCGGAAGGAGGAT |
| TaFAD6-R (SEQ ID NO: 7) | TCCGTCAGCTCAGCTTTGGCA |
| pinb-F (SEQ ID NO: 8) | AGGCAGGCTCGGTGGCTTCT |
| pinb-R (SEQ ID NO: 9) | TCGGCGCCCATGTTGCACTT |

C)

| Reaction composition | 1x (μL) |
|---|---|
| H2O | 8 |
| 2x iTaq ™ Universal SYBR ® Green | 10 |
| Mix of F and R Primers (10 μM | 0.5 |
| of each) | |
| DNA (12.5-50 ng/L) | 1.5 |
| Total | 20 |

Empirical validation of the specificity of the primer pairs was performed by melting curve analysis of the amplification products.

Raw data were processed by the CFX manager 3.1 software application (BioRad) using the settings baseline subtraction and linear regression, to obtain the Cq (Cycle Quantification) values.

For each primer pair, qPCR efficiency was also calculated by CFX manager software by the dilution method from data of three 1:2 serial dilutions of one of the T-generation T258 (T258-2) samples. Amplifications were performed in duplicates and mean Cqs (MCq) was calculated for each sample and primer pair combination.

To calculate the ratio between target and the references genes including the efficiency correction a modified formula (Equation 1) derived from the equations published by Pfaffl (2001) was used for each sample,

$$Ratio = N \times \frac{{( {E}_{( ref )} )}^{M C {q}_{( ref )}}}{{( {E}_{( target )} )}^{M C {q}_{( target )}}} \qquad Equation 1$$

where N is the number of copies per haploid genome for each of the reference genes (N=1, for pinb, and N=3 for TaFAd).

For estimating the mean transgene copy number per haploid genome in T258 line, the geometric means and the geometric standard deviations of the five OA-TaFAd ratios and the five OA-pinb ratios were calculated.

Results of BLAST show that in the ICWGS assembly of Triticum aestivum there are no perfect sequence matches of the primer pairs out of the targeted genes (Table 7). As expected, there are no hits for the primer OAsense_i_R, designed for amplifying the UBI intron of Zea mays present in the RNAi fragment inserted in T258 line.

### Table 7:

| Analysis | | |
|---|---|---|
| BLASTN | Job 1: TaFad_e6_F | Done: 3 hits found |
| | Job 2: TaFad_e6_R | Done: 3 hits found |
| | Job 3: pinb_F | Done: 1 hit found |
| | Job 4: pinb-R | Done: 1 hit found |
| | Job 5: OAsense_i_F | Done: 1 hit found |
| | Job 6: OAsense_i_R | |

Ensembl Plants release 48 - August 2020 © EMBL-EBI

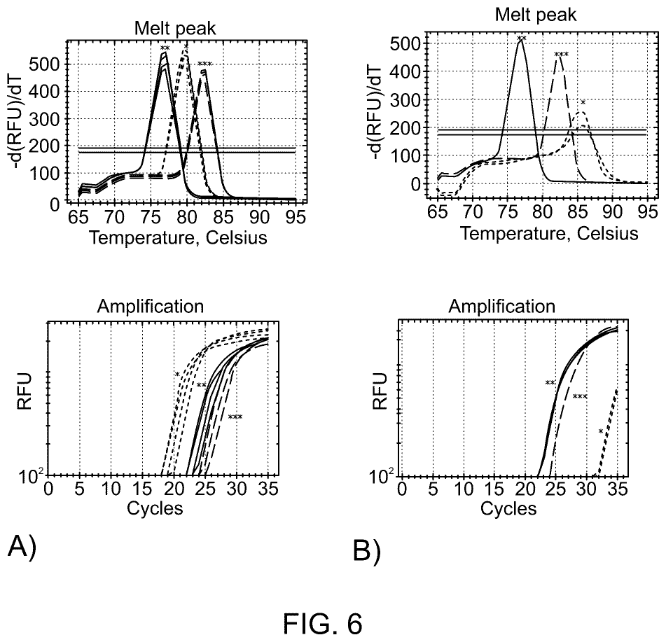

As shown in FIG. 6, Melting Analysis of the amplicons indicates that a specific product is amplified in each reaction for each primer pair. The mean melting temperatures (Tm) for the omega-alpha construct (OA), the three copies for the reference gene (TaFAd), and the single copy for the reference gene (pinb) are, respectively:
Tm_{OA}=79.6° C.
Tm_{pinb}=82.5° C.
Tm_{pinb}=77.0° C.

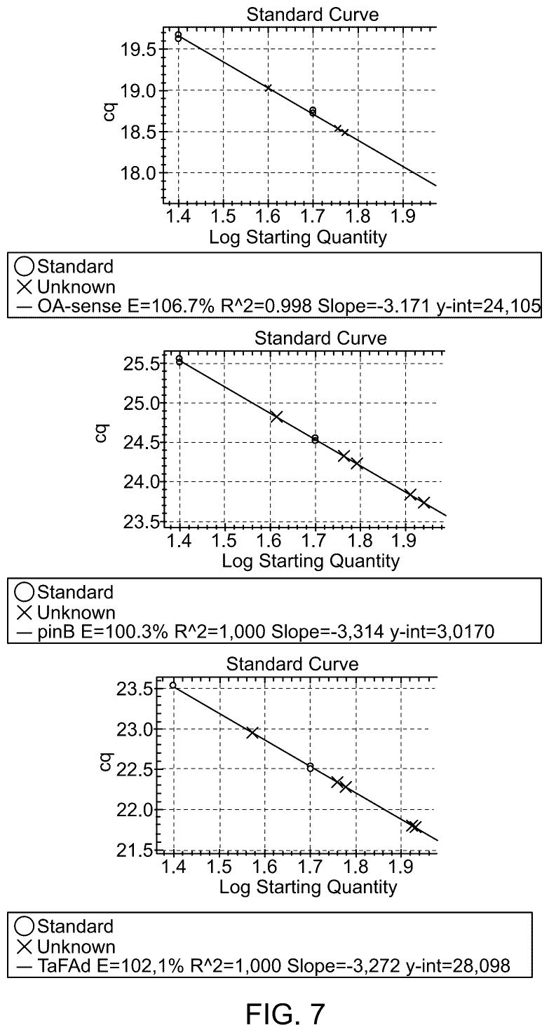

BW208 produces an non-specific amplification of OA target with a Tm=82.50° C. Standard curves were estimated for calculating the PCR efficiency for each primer pair (FIG. 7). Prior to using then on equation 1, the efficiency values estimated by the CFX software (E_{CFX}), expressed as a percentage, were transformed by the equation 2

$$E = \frac{{E}_{C F X}}{100} + 1$$

The transformed efficiency values were E_{OA}=2.067 (R²=0.998), E_{TaFAd}=2.021 (R²=1.000), and E_{pinb}=2.003 (R²=1.000) for the omega-alpha construct (OA), the three copies reference gene (TaFAd) and the single copy reference gene (pinb), respectively.

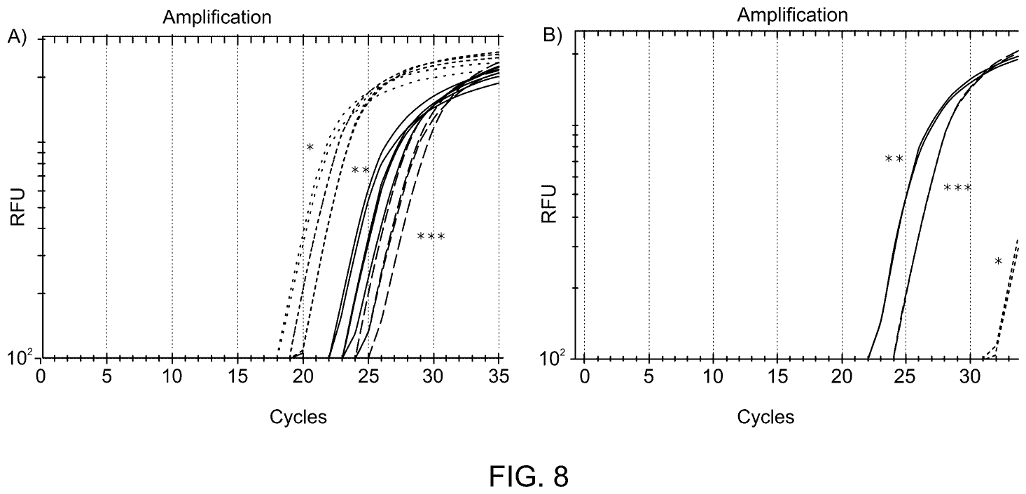

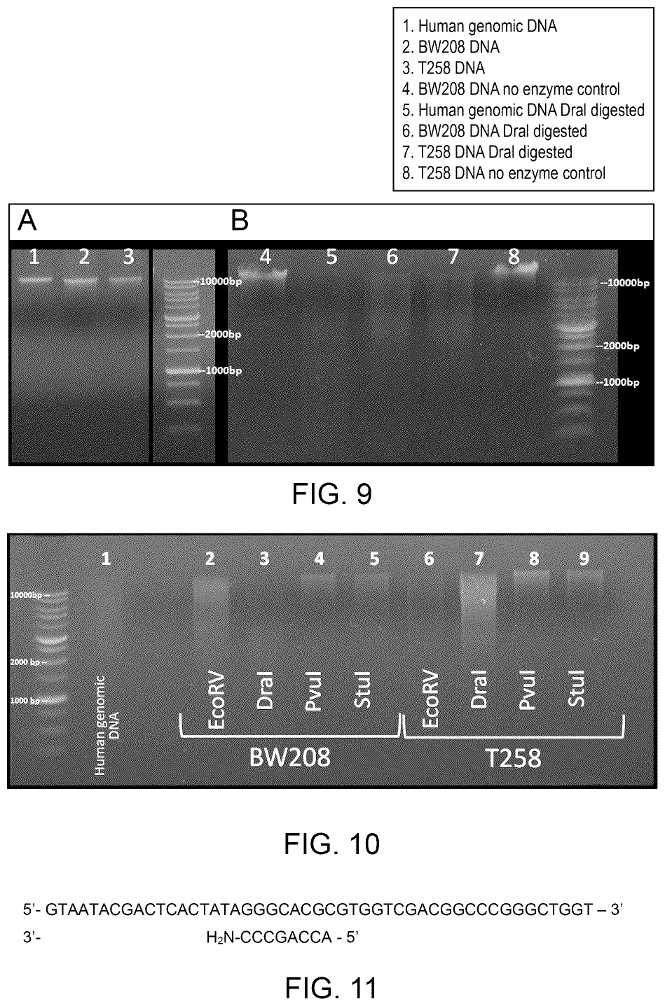

In FIG. 8, amplification curves of T258 and the wild type BW208 are shown. The wild type yields an amplification product, much later than the single copy gene reference pinB, confirming the amplification of an artifact. As a rule, a Cq greater than 30 is not considered as a reliable amplification of a DNA sequence (Table 9).

For each sample (or dilution) the OA/TaFAd, and TaFAd/pinb ratios were calculated according to equation 1 from the mean Cqs (Table 8) and the estimated efficiencies. The geometric mean of the OA/TaFAd ratios (29.30) and the OA/pinb ratios (31.31) are shown in Table 9. It is noteworthy that the TaFAd/pinb mean ratio value (Table 9) is approximately equal to 3, endorsing the three copies of the former gene as determined in silico.

TABLE 8
Mean quantification cycle (Cq) of the amplification
of OA-sense_i, TaFAd and pinb sequences for
each of the samples of the study for each.

| | Cq_{OA} | Cq_{TaFAd} | Cq_{pinb} |
|---|---|---|---|
| T₇ T258-1 | 19.03 | 22.95 | 24.82 |
| BW208 | 33.10 | 21.79 | 23.79 |
| T₇ T258-2 dil 1 | 17.74 | 21.56 | 23.54 |
| T₇ T258-2 dil 2 | 18.76 | 22.52 | 24.54 |
| T₇ T258-2 dil 3 | 19.65 | 23.53 | 25.54 |
| T₂ T258 | 18.52 | 22.31 | 24.28 |

TABLE 9
Number of copies (ratios) per haploid genome of each of the samples of the study.

| | | | | OA Mean |
|---|---|---|---|---|
| | Ratio_{OA-TaFAd} | Ratio_{OA-pinb} | Ratio_{TaFAd-pinb} | ratio |
| T₇ T258-1 | 31.00 | 30.83 | 2.98 | 30.91 |
| BW208 | NA | NA | 3.30 | |
| T₇ T258-2 dil 1 | 29.61 | 32.12 | 3.25 | 30.87 |
| T₇ T258-2 dil 2 | 27.77 | 30.86 | 3.33 | 29.31 |
| T₇ T258-2 dil 3 | 29.61 | 32.12 | 3.25 | 30.86 |
| T₂ T258 | 28.59 | 30.64 | 3.21 | 29.62 |
| Geometric mean* | 29.30 | 31.31 | 3.22 | 30.28 |
| Geometric SD* | 1.04 | 1.02 | 1.04 | 1.03 |
| Low limit = Mean/SD* | 28.11 | 30.58 | 3.10 | 29.51 |
| Upper limit = Mean × SD* | 30.53 | 32.06 | 3.35 | 31.08 |

*Geometric averaging is preferred to arithmetic averaging for managing qPCR data (Vandesompele et al., 2002). The geometric standard deviation is a multiplicative factor that describes how spread out is a set of numbers whose preferred average is the geometric mean.
In conclusion, for T258, the mean copy number of the RNAi silencing fragment quantified by qPCR is 30.28 copies per haploid genome.

### Example 7: Molecular Characterization of T258 RNAi Event

The determination of the integration site for the T258 RNAi event was conducted by DNA walking. DNA walking is a method for finding unknown genomic sequences adjacent (flanking sequences) to a known DNA sequence (insert). The technique was carried out using a commercial kit, the Universal GenomeWalker 2.0 kit (Takara Bio USA, Inc.) following manufacturer's instructions.

The kit uses an enzyme restriction-based approach that consists of adding specially synthesized DNA fragments with a known nucleotide composition (double-strand adapters) into the structure of linear DNA sequences. PCR amplification with adapters involves the sequential cleavage of genomic DNA by restriction enzymes frequently cutting which leaves blunt ends of digested DNA after the reaction. With a certain probability, genomic DNA is cut at a short distance from the border (a maximally effective distance depends on the nature of the polymerase used at the next stages). The blunt ends are subsequently ligated to the mixture of fragments of double-strand adapters with a known nucleotide sequence. Thus, it becomes possible to carry out a PCR reaction and to amplify an unknown fragment of genomic DNA with two specific primers, a Gene Specific Primer (GSP) one of which is annealed on the near-border of the known sequence, and a second primer (AP, Adaptor Primer) which is annealed on the adapter (AP, Adaptor Primer).

The unknown genomic sequences flanking the transgene are amplified by two consecutive PCR reactions (Nested PCR) using two different pairs of primers, one of which will be an AP and the other a GSP, minimizing amplification of unspecific products. Genomic sequences flanking the transgene are determined by sequencing the nested PCR products, so the identity of the integration site can be determined by genome database searches. In the case of integration if tandem-repeats of the transgene occurred; more than one cutting site of the restriction enzyme used for libraries preparation within the inserted fragment may exist, thus producing amplification products that will not be informative for the flanking sequences as they would contain exclusively the transgene sequence (Noguchi et al. 2004).

Genomic DNA was isolated from young leaves of T258 and BW208 (Wildtype, WT) plants using the CTAB method (Murray & Thompson, 1980) with minor modifications. To ensure that genomic DNA was of adequate quality for the following steps, 100 ng of genomic DNA and 1 μl of the human genomic DNA (0.1 μg/μl), provided with the kit as a control, were analyzed by 1% agarose gel electrophoresis showing the adequate molecular weight and minimum smearing (FIG. 10A). DNA purity and lack of inhibitor were check by Dral enzyme digestion (FIG. 10B). A control of the genomic DNA without any enzyme was also set up. Only Dral-digested genomic DNA produced a smear, indicating that it can be digested by restriction enzymes.

Next, separate samples of T258 and BW208 DNA were completely digested with four different frequently-cutting restriction enzymes, EcoRV, Dral, Stul and Pvul (Table 10) provided with the kit, which leaves blunt ends DNA after cutting. Additionally, one Pvull digestion of human genomic DNA was set up as a positive control. Five μl of each reaction were loaded in a 1% Agarose gel (FIG. 10) to ensure that the digestion was complete. Each batch of digested genomic DNA was then purified by the NucleoSpin Gel and PCR Clean-Up kit (Takara Bio USA, Inc.) following manufacturer's indications. After purification each sample was ligated to the Genome Walker Adaptor (FIG. 11; SEQ ID NO: 10) following supplier instructions.

TABLE 10
Blunt end restriction enzymes used for Genome
Walking “libraries”

| Restriction | |
|---|---|
| endonuclease | Recognition sequence (5′-3′) |
| EcoRV | GAT^{∧}ATC |
| DraI | TTT^{∧}AAA |
| StuI | AGG^{∧}CCT |
| PvuI | CGAT^{∧}CG |

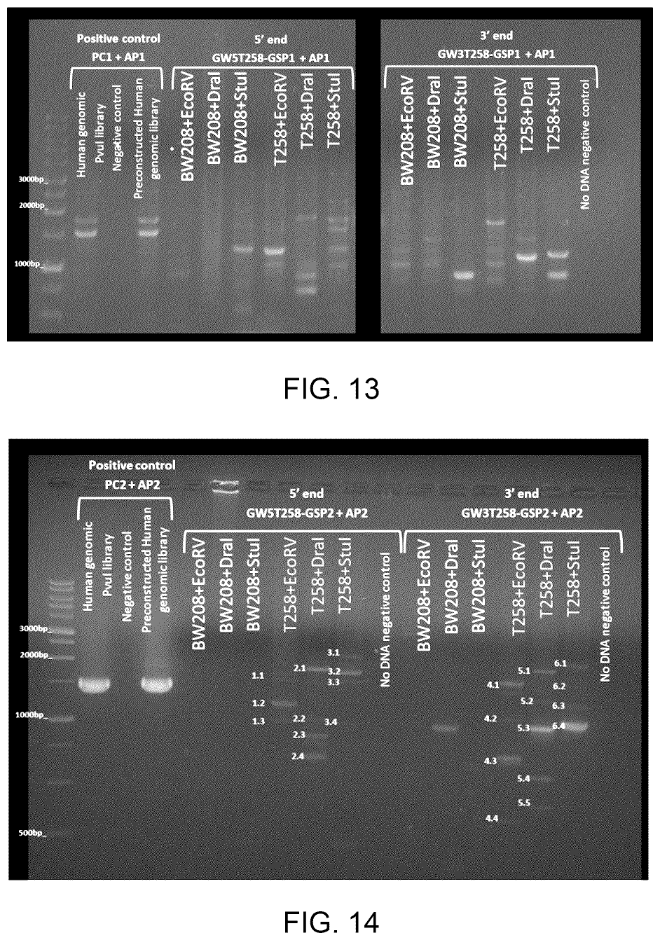

For genomic walking of the T258 RNAi event, two Gene-Specific Primers (GSP)—one for primary PCR (GSP1) and one for secondary PCR (GSP2)—for each flank, were designed (FIG. 23). The nested PCR primer (GSP2) should anneal to sequences beyond the 3′ end of the primary PCR primer (GSP1) without overlapping. Given the general consideration that the GSP should be derived from sequences as close to the border of the known sequence as possible, the regions upstream the gamma-gliadin promoter and downstream NOS terminator of the transgene were considered the best option for primer annealing sites. These sequences correspond to the expression vector back-bone, thus they are not part of the wheat genome background. Primer annealing with several elements of the fragment intended to be inserted that are already present in the wheat genetic background (gliadin promoter and gliadins sense and antisense sequences) was avoided as it could produce unwanted amplification products. Two GSP were designed to act as reverse primers for the amplification of the 5′ end of the transgene, GW5T258-GSP1 (SEQ ID NO: 11) and GW5T258-GSP2 (SEQ ID NO: 12), and two other primers were designed to act as forwards for the amplification of the 3′ end of the transgene, GW3T258-GSP1 (SEQ ID NO: 13) and GW3T258-GSP2 (SEQ ID NO: 14) (FIG. 13, Table 10). Pairing primers for the GSPs are provided by the Universal GenomeWalker 2.0 kit (AP1 (SEQ ID NO: 15) and AP2 (SEQ ID NO: 16) (Table 10)) and anneals with the Genome Walker Adaptor (FIG. 11; SEQ ID NO: 10).

TABLE 11
Primers used for Genome walking

| Primer Name | Annealing location | Sequence 5′-3′ |
|---|---|---|
| GW3T258-GSP1 | Downstream NOS terminator | GCATAAAGTGTAAAGCCTGGGGTGCCTA |
| GW3T258-GSP2 | Downstream NOS terminator | GCCAGCTGCATTAATGAATCGGCCAAC |
| GW5T258-GSP1 | Upstream Gamma-gliadin | TTTACAACGTCGTGACTGGGAAAACCCT |
| | promoter | |
| GW5T258-GSP2 | Upstream Gamma-gliadin | TACCCAACTTAATCGCCTTGCAGCACAT |
| | promoter | |
| AP1 | Genome Walker Adaptor | GTAATACGACTCACTATAGGGC |
| AP2 | Genome Walker Adaptor | ACTATAGGGCACGCGTGGT |

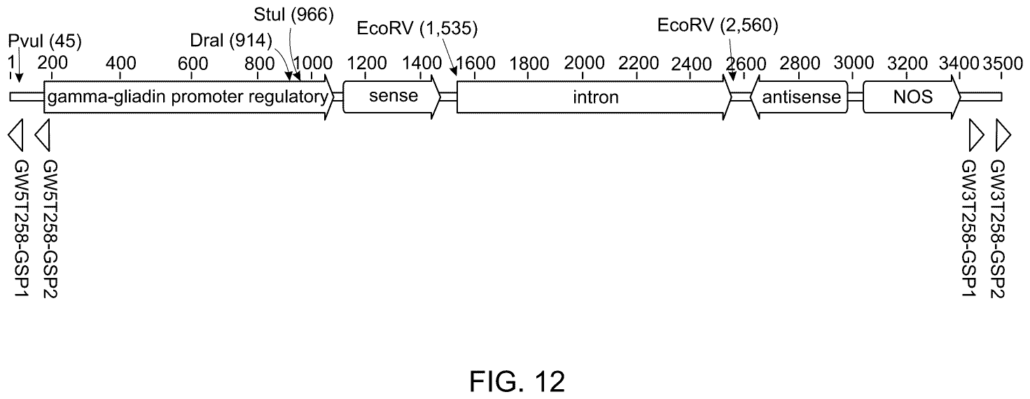

The Pvul Genome Walker libraries were discarded for PCR-based genomic walking as there was an intervening restriction site between the Gene-Specific Primers and the genomic flanking sequence in the 5′ end of the RNAi insert (FIG. 12).

Two PCR amplifications per library (FIG. 13) were carried out with the Advantage 2 Polymerase Mix included in the Genome Walking kit for what the cycling parameters in this protocol have been optimized. The Advantage 2 PCR Kit contains a polymerase mix suitable for long-distance PCR to extends the range of possible PCR products to around 6 kb, by using a combination of two thermostable DNA polymerases that increases the range and accuracy of PCR amplification, with an estimated error rate of 25 errors per 100000 bp after 25 PCR cycles. The polymerase mix include the Titanium® Taq DNA Polymerase from Takara Bio USA, Inc., and a minor amount of a proofreading polymerase. Most of the PCR extension is carried out by a primary polymerase, while a secondary polymerase provides the critical 3′ to 5′ exonuclease or “editing” function that corrects misincorporated nucleotides.

The first or primary PCR uses the outer adaptor primer (AP1) provided in the kit and one of the Gene-Specific Primer designed (GW5T258-GSP1 and GW3T258-GSP1, Table 10, for amplification of the 5′ and 3′ flanking sequence, respectively). One purified Pvul library from human genomic DNA (provided with the kit), plus the Pvul library from undigested control human genomic DNA (that was set up at the previous step as a control for the entire process of restriction digestion and adaptor ligation) were used as positive controls to test the system by amplification with the positive control gene-specific primers (PCP1 and PCP2, provided with the kit). Primary PCR products from the three BW208 Genome Walker libraries, three T258 Genome Walker libraries and two human genomic libraries amplified with their corresponding primer pairs were analyzed on a 1% agarose gel electrophoresis (FIG. 13). As showed, multiple bands were visible in the BW208 wildtype and T258 samples, but bands from BW208 samples were notably less numerous than from T258, and may correspond to unspecific amplifications that commonly occurs. Positives and negatives controls PCR products were as expected.

The primary PCR product mixture is then diluted and used as a template for a secondary or “nested” PCR with the nested adaptor primer (AP2) and a nested Gene-Specific Primers (GW3T258-GSP2 and GW5T258-GSP2). Single major bands were observed with each of the three libraries from T258.

Major bands observed in secondary PCR from T258 samples (numbered in FIG. 14) were separately excised from the gel and purified with by the NucleoSpin Gel and PCR Clean-Up kit (Takara Bio USA, Inc.) following supplier instructions. Each purification product was sequenced by Sanger method using AP2 and the corresponding GW3T258-GSP2 and GW5T258-GSP2 primers. Sequencing was carried out by Stab Vida (Caparica, Portugal). Table 11 summarizes the number of bands excised from each library amplification and the number of sequencing reads obtained. Geneious software (Geneious Prime® 2019.0.4, Biomatters Ltd., Auckland, New Zealand) was used for processing raw sequencing data. Due to the possible presence of more than one PCR product in some of the bands or to insufficient DNA template, many of the sequencing reads were empty or without enough quality, thus they were discarded as they do not provide any information. Low quality ends or regions with more than a 5% chance of an error per base were trimmed and resulting sequences were mapped to the transgene sequence. Most of the resulting sequences mapped within the transgene sequence, not being informative for the genomic flanking sequences. Some of the resulting sequences revealed the presence of head-to-tail tandem-repeats insertion of the transgene (for example those coming from PCR bands 1.2, 4.3, 5.3 or 5.4, FIG. 14) with a perfect match to the sticky ends produced by Haell restriction enzyme used for the insert production to release it from the plasmid.

TABLE 12
Number of bands and reads derived from Genome Walking of T258 wheat line.

| | Number of | | Not mapping |
|---|---|---|---|
| T258 Genome Walker | major | Resulting | with |
| Libraries Nested | bands | sanger | RNAi |
| PCR Product | excised | reads | insert |
| From 5′ flank amplification of the T258 EcoRV library | 3 | 6 | 1 |
| From 5′ flank amplification of the T258 Dral library | 4 | 5 | 0 |
| From 5′ flank amplification of the T258 Stul library | 4 | 2 | 0 |
| From 3′ flank amplification of the T258 EcoRV library | 4 | 3 | 0 |
| From 3′ flank amplification of the T258 Dral library | 5 | 4 | 1 |
| From 3′ flank amplification of the T258 Stul library | 4 | 5 | 0 |

Only two of the obtained Sanger reads revealed a fragment that do not mapped with the transgene sequence. The first sequence, of 845 bp in length, was obtained from a band purified from 5′ flank PCR amplification of the EcoRV digested T258 DNA library (band 1.1). The sequence of this fragment is as follows (SEQ ID NO: 17):

5′-TGCAAGAGATCGAGCGCGCTCTCCGCACGGTACGACGATGCGTGCC
GGAAGGAAGTGCGGTGCATCTTCCCAATGGTGCAGCCCTCGAAGAACTG
GTCGAGGTGCGCGATTGCTGGCATGCCGCGCACCATGCCCTTTGCACCG
AGGTCATGCAGCGCGCGGAAATGCAGATGCCCGAATCTTGCATGCCACC
TCCAGGCGTCGTCCTGAACTCCGGCGAGGAGGCACACGGGTATGGCCAA
GTTCAGAGCCACGGCGTAAAGCCGGTTGCTCGTCCGCCGCACCCGCGCG
AGTAGCAACCTCTGCCTGTCATGCAGGCAGAGTTCCCCATCCTCGATCA
CGGCTTTGCAGCCGTGCTCATCGAGCTGCCCGACGCTGACGATGCTTGT
GCGTAGCTGCTGGATGTAGTAGACGTTGGCGAGCGCGCGATGTTCACCG
TTGCGGCACGTGAAGAGCACGACGCCGCACCCACAAATCTGCACGACCG
AGCCGTCACCAAAACATACAGTACCTCGTACCGTCTCGTCGAGCTCCGA
GAGCATGTCGCGGCGCCCTGTCATGTGATTGCTAGCGCCGGTGTCGAGG
TACCACCGGTCGTCGTCCGTCGCCACGGGCACGACCCGTGCTTCGTTGA
GGAAGATCTCCATGCAGTCTGTTTCGCAATCTGTGTCAATTGGCCCAGC
TATCCTGTGGGCTGCTGCTCTATGGGCCGAATCCGAGGCCTCTGGTTAA
TTGGGCCGTCGGCCAGCCCATTCCAGCGCATCGCTAAAAGATGTCGCCA
TTCGCCATTCAGGCTGCGCAACTGTTGGGAAGGGCGATCGGTGCGGGCC
TCTTCGCTATACGCC-3′

The 69 bp marked in bold letter correspond to the 5′ border of the transgene, and the remaining 776 bp were considered as the potential unknown flanking target sequence close to the gamma-gliadin promoter.

A second sequence of 355 bp was obtained from a band amplified from the 3′ flank PCR amplification of the Dral digested T258 DNA library (band 5.5). The sequence of this fragment is as follows (SEQ ID NO: 18):

5′-TGCCAGCTGCATTAATGAATCGGCCAACGCGCGGGGAGCTCCTAGT
CGGCGCCTTAAGCGCCGGCTAGGAGAAGCAGCGCCCACGCGCCCTCCAA
GTGGGCCGGCCCATGAAACGCGTCGTACGCGAAAACCCACCAAAAAAGA
GGATACTACGGGATTCGAACTAAGGATGTTCACTCCACGTGCAAAGCAA
GCTGACCACTTGAGCTACCGACCGAGAGTGATTACAAACAGCGTGACAC
TGTAAATAACATTGTCGCGGCGCTACTGTTGCACATAACAATGTACTAT
ACATCGCATCAAATATTTGGGAACAACAAAAATCGTGACTAGAAAAGGT
TCATGGATTTGAAAA-3′

From which 38 bp, marked in bold, correspond to the 3′ border of the transgene and the remaining 317 bp were considered as the potential unknown flanking target sequence close to the NOS terminator of the transgene.

When mapping these GW derived sequences (SEQ ID NO: 17 and SEQ ID NO: 18) with the initial fragment intended to be inserted (SEQ ID NO: 1), we observed that one bp is missing from the 5′ end of the insert, while at the 3′ end there are 22 bp that are missing, thus were not inserted (SEQ ID NO: 32) (5′-AGGCGGTTTGCGTATTGGGCGCGC-3′).

These two putative flanking sequences were used for a BLAST search (excluding the sequences marked in bold) at the web-based bioinformatics ENSEMBL (www.plants.ensembl.org) to check if they correspond to the wheat genomic background to identify the insert integration site. As BW208 wheat cultivar genome is not sequenced, Blast was performed against all the Triticum aestivum, durum and urartu varieties reference genomes that are available, as well as scaffolded assemblies, in order to found similarities. Blast results as indicate In Table 13.

TABLE 13
Blast results for the Genome walking derived reads.

| Job | Genomic Location | Overlapping Gene(s) | Orientation | Length | Score | E-val | % ID |
|---|---|---|---|---|---|---|---|
| Triticum | 4A: 716889556- | | Forward | 774 | 774 | 0.0 | 100.0 |
| turgidum | 716890329 | | | | | | |
| Svevo.v1 | | | | | | | |
| Triticum | 7A: 7361722- | | Forward | 649 | 465 | 0.0 | 92.9 |
| turgidum | 7362370 | | | | | | |
| Svevo.v1 | | | | | | | |
| Triticum | 2B: 758710103- | TRITD2Bv1G253830 | Forward | 645 | 425 | 0.0 | 91.5 |
| turgidum | 758710747 | | | | | | |
| Svevo.v1 | | | | | | | |
| Triticum | Un: 121348374- | | Forward | 645 | 409 | 0.0 | 90.9 |
| turgidum | 121349018 | | | | | | |
| Svevo.v1 | | | | | | | |
| Triticum | cle_scaffold_{—} | | Reverse | 774 | 774 | 0.0 | 100.0 |
| aestivum Claire | 009109: 86864-87637 | | | | | | |
| Elv1.1 | | | | | | | |
| Triticum | cle_scaffold_{—} | | Forward | 645 | 401 | 0.0 | 90.5 |
| aestivum Claire | 093582: 30708-31352 | | | | | | |
| Elv1.1 | | | | | | | |
| Triticum | cle_scaffold_{—} | | Reverse | 645 | 441 | 0.0 | 92.1 |
| aestivum Claire | 071581: 14255-14899 | | | | | | |
| Elv1.1 | | | | | | | |
| Triticum | cle_scaffold_{—} | | Reverse | 645 | 465 | 0.0 | 93.0 |
| aestivum Claire | 002892: 170955-171599 | | | | | | |
| Elv1.1 | | | | | | | |
| Triticum | 7A: 8753131- | | Forward | 649 | 465 | 0.0 | 92.9 |
| aestivum | 8753779 | | | | | | |
| IWGSC | | | | | | | |
| Triticum | 2A: 15086519- | | Reverse | 645 | 465 | 0.0 | 93.0 |
| aestivum | 15087163 | | | | | | |
| IWGSC | | | | | | | |
| Triticum | 7B: 4623415- | | Forward | 645 | 429 | 0.0 | 91.6 |
| aestivum | 4624059 | | | | | | |
| IWGSC | | | | | | | |
| Triticum | 3A: 11522763- | | Reverse | 645 | 469 | 0.0 | 93.2 |
| aestivum | 11523407 | | | | | | |
| IWGSC | | | | | | | |
| Triticum | par_scaffold_{—} | | Forward | 774 | 774 | 0.0 | 100.0 |
| aestivum | 012658: 78708-79481 | | | | | | |
| Paragon Elv1.1 | | | | | | | |
| Triticum | par_scaffold_{—} | | Forward | 645 | 437 | 0.0 | 91.9 |
| aestivum | 007793: 169146-169790 | | | | | | |
| Paragon Elv1.1 | | | | | | | |
| Triticum | par_scaffold_{—} | | Reverse | 645 | 425 | 0.0 | 91.5 |
| aestivum | 011077: 101223-101867 | | | | | | |
| Paragon Elv1.1 | | | | | | | |
| Triticum | par_scaffold_{—} | TraesPAR_scaffold_{—} | Reverse | 645 | 497 | 0.0 | 94.3 |
| aestivum | 097893: 29777-30421 | 097893_01G000100 | | | | | |
| Paragon Elv1.1 | | | | | | | |

Blast output data (Table 13) with higher identity (100% ID) was retrieved and used to reconstruct the potential insertion site (FIG. 16). From Blast output information, we deduced that the transgene must have been inserted in reverse orientation with respect to the best matching wheat genome references, thus from this point onwards we will refer as the 5′ end or flank to indicate flanking sequence close to the NOS terminator of the insert (i.e. containing flank sequence upstream the insert) and the 3′ end to indicate flanking sequence close to the Gamma-gliadin promoter (i.e. the flank sequence downstream the insert).

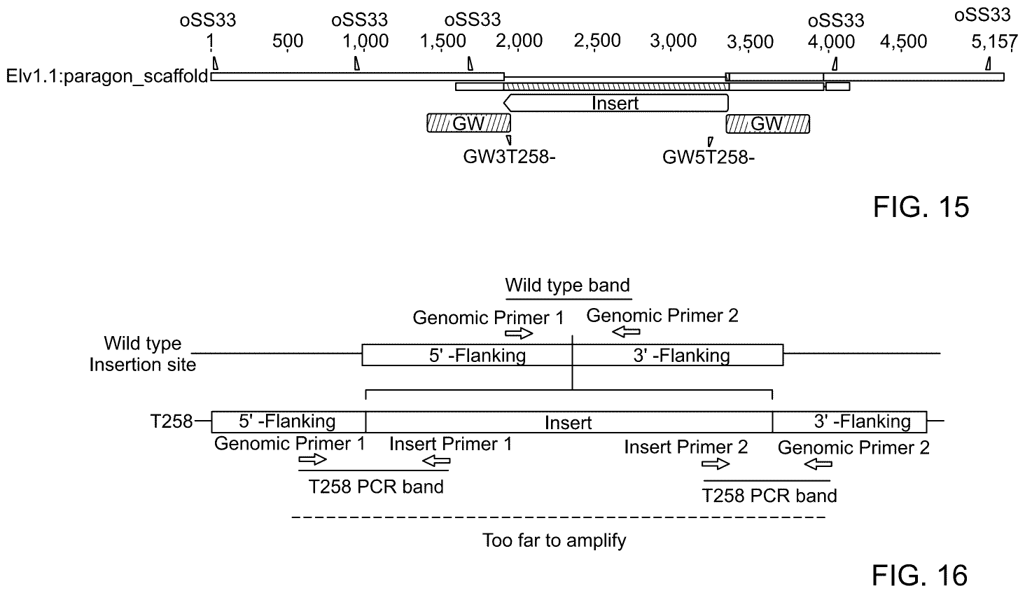

To verify that the genomic flanking sequences obtained by Sanger of the Genome Walking products were not an artefact, and that they are in fact present in T258 event, we designed a couple of primers that anneals in them (primers oSS337 and oSS334, annealing in the 5′ and 3′ flanks, respectively, Table 14, FIG. 15). Also, to check that the wheat genome reference sequences obtained by the Blast of Genome Walking Sanger results correspond to the insertion site of the transgene, a set of primers were designed to anneal upstream (primers oSS338 and oSS339, Table 14) and downstream (oSS333, Table 14) the Genome walking sequences (FIG. 15).

TABLE 14
Primers annealing the genomic flanking sequence

| Primer | | |
|---|---|---|
| Name | Position | Sequence 5′-3′ |
| oSS333 | Downstream the insertion, based on blast data | ACTGGCACTTGCTGA |
| | | TGCT |
| oSS334 | Downstream the insertion, based on flanking sequence | GTGCAGCCCTCGAA |
| | obtained by GW | GAACT |
| oSS337 | Upstream the insertion, based on flanking sequence | GCGCCGCGACAATG |
| | obtained by GW | TTATT |
| oSS338 | Upstream the insertion, based on blast data | CCCTTAAAGGCCAAC |
| | | CCGT |
| oSS339 | Upstream the insertion, based on blast data | CATCGATGGCTCAG |
| | | GCGAT |

TABLE 15
Primers annealing at the transgene

| Primer Name Position | Sequence 5′-3′ |
|---|---|
| pDhpA/BZR- | CCAGTGCCAAGCTTGCATG |
| F1 transgene | |
| pDhpA/BZR- | GCACCCCAGGCTTTACACT |
| R1 transgene | |
| pDhpA/BZR- | AACACCCCCATGTCATGCT |
| R2 transgene | |
| pA/BZR-NOS- | CGCGCGGTGTCATCTATGT |
| F2 transgene | |

PCR amplifications of T258 and the wild type BW208 (as a negative control) were carried out combining one of the primers that anneals in the transgene (Table 15 and GSPs from Table 11) with one of the new primers annealing in the genomic flanking sequence (Table 14). PCR reactions produced a clear band of the expected size in T258 (FIG. 16) but did not amplify in BW208 (wild type). These results confirmed that the sequence data of the insertion site obtained by Genome Walking (i) were not an artefact, (ii) is located in the Triticum genomic region obtained by Blast and are specific from the T258 event, not being present in the BW208 wild type. On the other hand, PCR combining primers that anneals in both sides of the genomic sequence out of the insertion site (i.e., oSS333 or oSS334 with oSS37-39) only amplify a band of the expected size in BW208 (wild type), with no amplification in T258. These results corroborate the presence of the intact insertion site in the wildtype, meanwhile the length of the insert interrupting the insertion site produce no amplification in T258 (FIG. 16).

TABLE 16
Details of pairs of primers used for amplification and Sanger sequencing of the flanking
sequences in T258 and the intact corresponding insertion site in BW208 wildtype.

| | | | | Expected |
|---|---|---|---|---|
| | | | Amplification | Size |
| Target | Forward | Reverse | efficiency | (bp) |
| 5′ flank | oSS337 | GW3T258-GSP1 | High | 366 |
| 5′ flank | oSS337 | pA/BZR-NOS-F2 | High | 494 |
| 5′ flank | oSS338 | pA/BZR-NOS-F2 | Low | 1228 |
| 5′ flank | oSS338 | GW3T258-GSP1 | Only with Pfu polimerase | 1100 |
| 5′ flank | oSS339 | pA/BZR-NOS-F2 | Only with Pfu polimerase | 2128 |
| 3′ flank | GW5T258- | oSS334 | High | 853 |
| | GSP1 | | | |
| 3′ flank | pDhpA/BZR-R2 | oSS334 | High | 1100 |
| 3′ flank | GW5T258- | oSS333 | Only with Pfu polimerase | 1848 |
| | GSP1 | | | |
| 3′ flank | pDhpA/BZR-R2 | oSS333 | Only with Pfu polimerase | 2092 |
| Wildtype insertion site | oSS334 | oSS337 | High | 950 |
| Wildtype insertion site | oSS333 | oSS337 | Only with Pfu polimerase | 2000 |
| Wildtype insertion site | oSS334 | oSS338 | Only with Pfu polimerase | 1700 |

Sanger sequencing of the PCR products from mentioned amplifications (Table 16) was processed as previously described, discarding low quality data, and was assembled to expand the information and length of the flanking sequences in T258 and of the insertion site in the BW208 wildtype. Processing of the sequencing data yielded the following consensus sequences:

5′ flanking sequence in T258 (1124 bp)
SEQ ID NO: 28
CCCATTTCATTTACTTTTTTCTGTCAGCAAAAAGTTCGCTGGAGAGGCCAACCCAG
GACTTCCTTTGTTTAGCGTGAATGCAATGACCAACCAGCCACCCTCACCTAGTGT
GACGGGTTCCATCTTTTTTTCTATTGTCTTCTTTCTTCTTTTTTCTTCTGTACGTTTT
CTTTCGTCTTTTCGTTTTTTTTCTGTACCTTTTCTCAAAGAATGCGTGAATTTGTTTT
TTGTAATATCCGTTGAACATTTTGCGATATCCGTTGACATTTTCTTGAAATAAGCTG
AATATTTTAAATAATACATGGGGAACTTATTTTAATATACGATGAACATTTTTTAAAA
AATAGGAATATATGCGCTGAAGAATTTTTGGACACGTACTGAACATTTTGTGAAAA
TATGTTGAACAATTTGTGAATACACATTGAACATTGTGTAATATATACTGAACAAAA
TTAAGAAACAGAGTGAACAATTGTATACTCATTGAATAACCAAAACCAAAATAGAAA
AAAAAAATCAAAAAATATGTATATATATATCCTGAACATTTTTATATCCATTGAACAT
TTTAAATAATACACGATGAACATTTTCAAAATCCATGAACCTTTTCTAGTCACGATT
TTTGTTGTTCCCAAATATTTGATGCGATGTATAGTACATTGTTATGTGCAACAGTAG
CGCCGCGACAATGTTATTTACAGTGTCACGCTGTTTGTAATCACTCTCGGTCGGTA
GCTCAAGTGGTCAGCTTGCTTTGCACGTGGAGTGAACATCCTTAGTTCGAATCCC
GTAGTATCCTCTTTTTTGGTGGGTTTTCGCGTACGACGCGTTTCATGGGCCGGCC
CACTTGGAGGGCGCGTGGGCGCTGCTTCTCCTAGCCGGCGCTTAAGGCGCCGA
CTAGGAGCTCCCCGCGCGTTGGCCGATTCATTAATGCAGCTGGCACGACAGGTT
TCCCGACTGGAAAGCGGGCAGTGAGCGCAACGCAATTAATGTGAGTTAGCTCA
CTCATTAGGCACCCCAGGCTTTACACTTTATGCTTCCGGCTCGTATGTTGTGTGG
AATTGTGAGCGGATAACAATTTCACACAGGAAACAGCTATGACATGATTACGAA
TTCTG
Sequence corresponding with the insert is represented in bold
3′ flanking sequence in T258 (1781 bp)
SEQ ID NO: 29
ATCATAATCATTTTACTAATCCTCTTTCCGTTTCAAACCAAAACTCAAATCCAAAT
TCGAATGCCAAGTCGAATTATAATTTTATAAAAAATAAATTAAAAATGTTCCTTA
GGTTGAAAATATTCACTACATAACTGTCATACATTAGCAAAGTTTTTTCTGGAAG
CATGCAAGCTTGGCACTGGCCGTCGTTTTACAACGTCGTGACTGGGAAAACCCT
GGCGTTACCCAACTTAATCGCCTTGCAGCACATCCCCCTTTCGCCAGCTGGCGT
AATAGCGAAGAGGCCCGCACCGATCGCCCTTCCCAACAGTTGCGCAGCCTGAA
TGGCGAATGGCGACATCGTTTAGCGATGCGCTGGAATGGGCTGGCCGACGGCC
CAGTTAACCAGAGGCCTCGGATTCGGCCCATAGAGCAGCAGCCCACAGGATAGC
TGGGCCAATTGACACAGATTGCGAAACAGACTGCATGGAGATCTTCCTCAACGAA
GCACGGGTCGTGCCCGTGGCGACGGACGACGACCGGTGGTACCTCGACACCGG
CGCTAGCAATCACATGACAGGGCGCCGCGACATGCTCTCGGAGCTCGACGAGAC
GGTACGAGGTACTGTATGTTTTGGTGACGGCTCGGTCGTGCAGATTTGTGGGTGC
GGCGTCGTGCTCTTCACGTGCCGCAACGGTGAACATCGCGCGCTCGCCAACGTC
TACTACATCCAGCAGCTACGCACAAGCATCGTCAGCGTCGGGCAGCTCGATGAG
CACGGCTGCAAAGCCGTGATCGAGGATGGGGAACTCTGCCTGCATGACAGGCAG
AGGTTGCTACTCGCGCGGGTGCGGCGGACGAGCAACCGGCTTTACGCCGTGGC
TCTGAACTTGGCCATACCCGTGTGCCTCCTCGCCGGAGTTCAGGACGACGCCTG
GAGGTGGCATGCAAGATTCGGGCATCTGCATTTCCGCCGCTGCATGACCTCGGT
GCAAAGGGCATGGTGCGCGGCATGCCAGCAATCGCGCACCTCGACCAGTTCTTC
GAGGGCTGCACCATTGGGAAGATGCACCGCACTTCCTTCCGGCACGCATCGTCG
TACCGTGCGGAGAGCGCGCTCGAGCTCGTGCACTGGCGACTTGTGTGGTCCGAT
CACTCCGGCGACGGCCAGCGGTAACAAGTATTTTCTATTGATAGTTGACGATTTCA
GCAGATACATGTGGCTTGAGGTACTGAAAAGCAAGGATGAGGCATTTCGGTACTT
CAAGAAGATCAAGGCGCACGCGGACATACAAGGAAACGCCAAGCTCCGGGCGTT
CCACACAGACAGAGGTGGCGAGTTCACCTCGAACGAATTCGCCGCCTACTGTGA
AGAACTGGGCATCCAGAGGAACACGACGACGCCGTACTCTCCTCAACAAAATGGA
GTGGTCGAACGGAGAAACCAGATTGTCGGGGAGATGGCCCACAGTCTCATGAAG
GCCATGGGCGTGCCGTGCGCGTTCTGGGGCGAGGCCGTGCGCACCGCCGTCCA
CATCCTCAATCGGTCTCCAACCAGAAGCCTGCAGGGCATCACGCCATACGAGAC
GTGGCGCAAGAAGAAGCCAGCGGTGGACTACTTCCGCACCTTCGGCTGCGTCGT
GCATGTCAAGCTCATCGGACCAAGAGTGACAAAGCTGGCGGACAGATCGCGTCC
TGGCATCTTCCCGGGGTACGAGCTGGGGACGAAGGGATATCAGGTGTACGACCC
GATTGACAAGCGTCTGTACATCACACATGATGTGCGCTTCGAGGAGGATCGCCGC
TGGGACTGGAGCAAGGACGCCGGCGACTCGATGCACACCTGTGAGTTCACTGTC
GTGTACTCCGACGCGGGTTCGCCCGTAAC
Sequence corresponding with the insert is represented in bold, of which
sequence corresponding with part of the Gamma-gliadin promoter of the
insert is represented in purple color.
BW208 (wild type) full sequence of the Insertion site
SEQ ID NO: 33
CCCTTAAAGGCCAACCCGTACAGGGCGCACACCCCCCTCTCCCCGCTGGGCCGG
CCCATTTCATTTACTTTTTTCTGTCAGCAAAAAGTTCGCTGGAGAGGCCAACCCAG
GACTTCCTTTGTTTAGCGTGAATGCAATGACCAACCAGCCACCCTCACCTAGTGT
GACGGGTTCCATCTTTTTTTCTATTGTCTTCTTTCTTCTTTTTTCTTCTGTACGTTTT
CTTTCGTCTTTTCGTTTTTTTTCTGTACCTTTTCTCAAAGAATGCGTGAATTTGTTTT
TTGTAATATCCGTTGAACATTTTGCGATATCCGTTGACATTTTCTTGAAATAAGCTG
AATATTTTAAATAATACATGGGGAACTTATTTTAATATACGATGAACATTTTTTAAAA
AATAGGAATATATGCGCTGAAGAATTTTTGGACACGTACTGAACATTTTGTGAAAA
TATGTTGAACAATTTGTGAATACACATTGAACATTGTGTAATATATACTGAACAAAA
TTAAGAAACAGAGTGAACAATTGTATACTCATTGAATAACCAAAACCAAAATAGAAA
AAAAAATCAAAAAATATGTATATATATATCCTGAACATTTTTATATCCATTGAACATT
TTAAATAATACACGATGAACATTTTCAAAATCCATGAACCTTTTCTAGTCACGATTT
TTGTTGTTCCCAAATATTTGATGCGATGTATAGTACATTGTTATGTGCAACAGTAGC
GCCGCGACAATGTTATTTACAGTGTCACGCTGTTTGTAATCACTCTCGGTCGGTAG
CTCAAGTGGTCAGCTTGCTTTGCACGTGGAGTGAACATCCTTAGTTCGAATCCCG
TAGTATCCTCTTTTTTGGTGGGTTTTCGCGTACGACGCGTTTCATGGGCCGGCCC
ACTTGGAGGGCGCGTGGGCGCTGCTTCTCCTAGCCGGCGCTTAAGGCGCCGACT
AGGAGCTCCCCGGGACTAGATGGATGATGTCGACATCGTTTAGCGATGCGCTGG
AATGGGCTGGCCGACGGCCCAGTTAACCAGAGGCCTCGGATTCGGCCCATAGAG
CAGCAGCCCACAGGATAGCTGGGCCAATTGACACAGATTGCGAAACAGACTGCAT
GGAGATCTTCCTCAACGAAGCACGGGTCGTGCCCGTGGCGACGGACGACGACC
GGTGGTACCTCGACACCGGCGCTAGCAATCACATGACAGGGCGCCGCGACATGC
TCTCGGAGCTCGACGAGACGGTACGAGGTACTGTATGTTTTGGTGACGGCTCGG
TCGTGCAGATTTGTGGGTGCGGCGTCGTGCTCTTCACGTGCCGCAACGGTGAAC
ATCGCGCGCTCGCCAACGTCTACTACATCCAGCAGCTACGCACAAGCATCGTCAG
CGTCGGGCAGCTCGATGAGCACGGCTGCAAAGCCGTGATCGAGGATGGGGAACT
CTGCCTGCATGACAGGCAGAGGTTGCTACTCGCGCGGGTGCGGCGGACGAGCA
ACCGGCTTTACGCCGTGGCTCTGAACTTGGCCATACCCGTGTGCCTCCTCGCCG
GAGTTCAGGACGACGCCTGGAGGTGGCATGCAAGATTCGGGCATCTGCATTTCC
GCGCGCTGCATGACCTCGGTGCAAAGGGCATGGTGCGCGGCATGCCAGCAATC
GCGCACCTCGACCAGTTCTTCGAGGGCTGCACCATTGGGAAGATGCACCGCACT
TCCTTCCGGCACGCATCGTCGTACCGTGCGGAGAGCGCGCTCGAKCTCKTGCAC
GGCGACTTGTGTGGTCCGATCACTCCGGCGACGGCCAGCGGTAACAAGTATTTT
CTATTGATAGTTGACGATTTCAGCAGATACATGTGGCTTGAGGTACTGAAAAGCAA
GGATGAGGCATTTCGGTACTTCAAGAAGATCAAGGCGCACGCGGACATACAAGG
AAACGCCAAGCTCCGGGCGTTCCACACAGACAGAGGTGGCGAGTTCACCTCGAA
CGAATTCGCCGCCTACTGTGAAGAACTGGGCATCCAGAGGAACACGACGACGCC
GTACTCTCCTCAACAAAATGGAGTGGTCGAACGGAGAAACCAGATTGTCGGGGAG
ATGGCCCACAGTCTCATGAAGGCCATGGGCGTGCCGTGCGCGTTCTGGGGCGAG
GCCGTGCGCACCGCCGTCCACATCCTCAATCGGTCTCCAACCAGAAGCCTGCAG
GGCATCACGCCATACGAGACGTGGCGCAAGAAGAAGCCAGCGGTGGACTACTTC
CGCACCTTCGGCTGCGTCGTGCATGTCAAGCTCATCGGACCAAGAGTGACAAAG
CTGGCGGACAGATCGCGTCCTGGCATCTTCCCGGGGTACGAGCTGGGGACGAA
GGGATATCAGGTGTACGACCCGATTGACAAGCGTCTGTACATCACACATGATGTG
CGCTTCGAGGAGGATCGCCGCTGGGACTGGAGCAAGGACGCCGGCGACTCGAT
GCACACCTGTGAGTTCACTGTCGTGTACTCCGACGCGGGTTCGCCCGTAACCAC
GACCTACACTGTGTCACCCGAGGTGGTGGGATCACCGCGAGCGCGTGCAACGCC
AACTACGCCAAGCACACCAGTTCCACTGCAATCCCCTCGGACGCCCGTGTCCGG
CGGGTCGGGCGTGGGCACGTCGAGTGCGTCGAGCGTGCCAGCATCAGCAAGTG
CCAGT

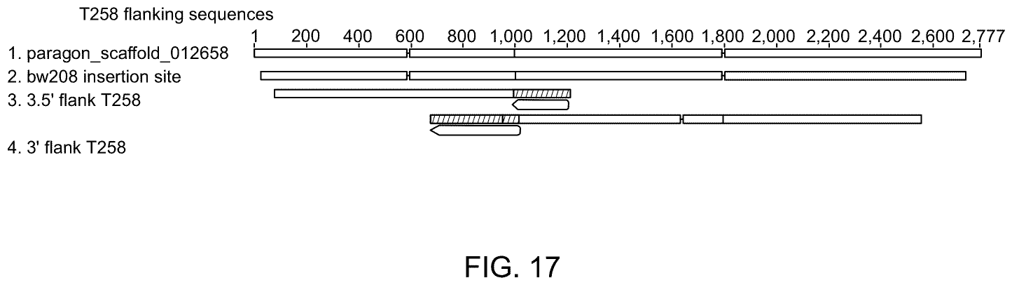

The comparison between the intact region of the BW208 wildtype and T258 flanking sequences (FIG. 17) showed that there are 18 bp in the wildtype that are not present in either of the flanking sequences, and maybe lost during insertion process. The 7 bp that appear just upstream the missing 18 bp, are present in both, the insertion site and the transgene sequences.

### Event-Specific PCR Detection

Based on the efficiency obtained of PCR amplification with a regular Taq polymerase, three pairs of primers have been selected (Table 17) as the best option for rapid event-specific PCR detection.

TABLE 17
Best primers for event specific PCR detection in T258

| | | | Amplification | |
|---|---|---|---|---|
| | | | efficiency | Product |
| | | | with Taq | Size |
| Target | Forward | Reverse | polimerase | (bp) |
| Terminator + | oSS337 | pA/BZR-NOS- | High | 494 |
| flanking sequence | | F2 | | |
| Promoter + flanking | pDhpA/BZR- | oSS334 | High | 1100 |
| sequence | R2 | | | |
| BW208 insertion | oSS334 | oSS337 | High | 950 |
| site | | | | |

### Chromosomal Mapping

The flanking genomic sequence information can serve in combination with genome database search for chromosomal mapping of the integration site of the intended fragment to be inserted. The Basic Local Alignment Search Tool (BLAST) finds regions of local similarity between sequences. BLAST uses statistical theory to produce a bit score and expect value (E-value) for each alignment pair (query to hit). The bit score gives an indication of how good the alignment is, the higher the score, the better the alignment. Blast results from Ensembl database are summarized in Table 18, where the best result from each job against a different available wheat variety reference is showed. A 100% ID match of the full sequence was obtained with some varieties of hexaploid wheat such as Paragon or Claire (Table 18). The genomic location of Blast output is annotated as a scaffold for most of the Blast results and, therefore, the chromosome of the insertion site cannot be determined. No 100% homology was found for the full sequence against the IWGSC reference genome, providing homology ranging from 88 to 93% for only fragments of up to 1570 bp, and mapping on chromosomes 1D, 7A, 3A, 3B, 7B, 5B and 7B. However, there is a clear match of the full sequence from the insertion site (2691 bp) with the 4A chromosome in durum wheat Svevo (Table 10). There is no information annotated in the proximity of this particular hit which means that the insertion is unlikely interrupting an important endogenous gene. However, there is a fragment of 1570 bp (from 2691 bp) from the BW208 insertion site that present a 93% of homology with the IWGSC reference genome in the 1D chromosome (1D: 208744361-208745930) where there is a putative gene (TraesCS1D02G150800).

TABLE 18
Blasts best results with 100% identity.

| | | Overlapping | | | | | |
|---|---|---|---|---|---|---|---|
| | Genomic | Gene(s) | | | | | |
| Job | Location | annotated | Orientation | Length | Score | E-val | % ID |
| Triticum aestivum | par_scaffold_{—} | — | Forward | 2691/ | 2691 | 0.0 | 100.0 |
| Paragon Elv1.1 | 012658: 77716- | | | 2691 | | | |
| | 80406 | | | | | | |
| Triticum aestivum | cle_scaffold_{—} | — | Reverse | 2691/ | 2691 | 0.0 | 100.0 |
| Claire Elv1.1 | 009109: 85939- | | | 2691 | | | |
| | 88629 | | | | | | |
| Triticum turgidum | 4A: 716888564- | — | Forward | 2691/ | 2683 | 0.0 | 99.9 |
| Svevo.v1 | 716891254 | | | 2691 | | | |
| Triticum | wee_scaffold_{—} | — | Reverse | 2128/ | 2128 | 0.0 | 100.0 |
| aestivum Weebill | 010497: 73157- | | | 2691 | | | |
| WeebilV1 | 75284 | | | | | | |
| Triticum aestivum | cad_scaffold_{—} | — | Forward | 2128/ | 2128 | 0.0 | 100.0 |
| Cadenza Elv1.1 | 015182: 86910- | | | 2691 | | | |
| | 89037 | | | | | | |

### REFERENCES

D'Haene B, Vandesompele J, Hellemans J. Accurate and objective copy number profiling using real-time quantitative PCR. Methods, 50 (2010), pp. 262-270
Li Z, Hansen J, Liu Y, Zemetra R, Berger P. Using real-time PCR to determine transgene copy number in wheat. Plant Molecular Biology Reporter (2004) volume 22, pages 179-188 (2004)
Pfaffl MW. A new mathematical model for relative quantification in real-time RT-PCR. Nucleic Acids Res (2001) 29: e45
Vandesompele J, De Preter K, Pattyn F, Poppe B, Van Roy N, De Paepe A, Speleman F. Accurate normalization of real-time quantitative RT-PCR data by geometric averaging of multiple internal control genes. Genome Biol. 2002 Jun. 18; 3 (7): RESEARCH0034. doi: 10.1186/gb-2002-3-7-research0034. Epub 2002 Jun. 18. PMID: 12184808; PMCID: PMC126239.
Murray, M. G., & Thompson, W. F. (1980). Rapid isolation of high molecular weight plant DNA. Nucleic Acids Research, 8 (19), 4321-4325. doi: 10.1093/nar/8.19.4321
Noguchi, A., Takekawa, N., Einarsdottir, T., Koura, M., Noguchi, Y., Takano, K., . . . . Suzuki, O. (2004). Chromosomal Mapping and Zygosity Check of Transgenes Based on Flanking Genome Sequences Determined by Genomic Walking. Experimental Animals, 53 (2), 103-111. doi: 10.1538/expanim.53.103

APPENDIX 1
Results from BLAST analysis (see Table 7)

1. Results for Job 1: TaFad_e6_F
BLASTN 2.9.0+
Reference: Stephen F. Altschul, Thomas L. Madden, Alejandro A.
Schaffer, Jinghui Zhang, Zheng Zhang, Webb Miller, and David J.
Lipman (1997), ″Gapped BLAST and PSI-BLAST: a new generation of
protein database search programs″, Nucleic Acids Res. 25: 3389-3402.
Database: Triticum_aestivum.IWGSC.dna.toplevel
22 sequences; 14, 547, 261, 565 total letters
Query = TaFad_e6_F
Length = 22

| | Score |
|---|---|
| E | (Bits) |
| Sequences producing significant alignments: | |
| Value | |
| EG:6D dna:chromosome chromosome:IWGSC:6D:1:473592718:1 REF | 44.1 |
| 0.003 | |
| EG:6B dna:chromosome chromosome:IWGSC:6B:1:720988478:1 REF | 44.1 |
| 0.003 | |
| EG:6A dna:chromosome chromosome:IWGSC:6A:1:618079260:1 REF | 44.1 |
| 0.003 | |

>EG:6D dna:chromosome chromosome:IWGSC:6D:1:473592718:1 REF
Length = 473592718
Score = 44.1 bits (22), Expect = 0.003
Identities = 22/22 (100%), Gaps = 0/22 (0%)
Strand = Plus/Minus

| | | |
|---|---|---|
| Query 1 | GCTTGGCATTCGGAAGGAGGAT | 22 |

||||||||||||||||||||||

| | | |
|---|---|---|
| Sbjct 46855672 | GCTTGGCATTCGGAAGGAGGAT | 46855651 |

>EG:6B dna:chromosome chromosome:IWGSC:6B:1:720988478:1 REF
Length = 720988478
Score = 44.1 bits (22), Expect = 0.003
Identities = 22/22 (100%), Gaps = 0/22 (0%)
Strand = Plus/Minus

| | | |
|---|---|---|
| Query 1 | GCTTGGCATTCGGAAGGAGGAT | 22 |

||||||||||||||||||||||

| | | |
|---|---|---|
| Sbjct 115946405 | GCTTGGCATTCGGAAGGAGGAT | 115946384 |

>EG:6A dna:chromosome chromosome:IWGSC:6A:1:618079260:1 REF
Length = 618079260
Score = 44.1 bits (22),
Expect = 0.003
Identities = 22/22 (100%), Gaps = 0/22 (0%)
Strand = Plus/Minus

| | | |
|---|---|---|
| Query 1 | GCTTGGCATTCGGAAGGAGGAT | 22 |

||||||||||||||||||||||

| | | |
|---|---|---|
| Sbjct 60443449 | GCTTGGCATTCGGAAGGAGGAT | 60443428 |

| | | |
|---|---|---|
| Lambda | K | H |
| 1.37 | 0.711 | 1.31 |

Gapped

| | | |
|---|---|---|
| Lambda | K | H |
| −1.00 | −1.00 | −1.00 |

Effective search space used: 58189044676
Database: Triticum aestivum. IWGSC.dna.toplevel
Posted date: Aug. 4, 2020 11:54 AM
Number of letters in database: 14, 547, 261, 565
Number of sequences in database: 22
Matrix: blastn matrix 1 −3
2. Results for Job 2. TaFad_e6_R
BLASTN 2.9.0+
Reference: Stephen F. Altschul, Thomas L. Madden, Alejandro A.
Schaffer, Jinghui Zhang, Zheng Zhang, Webb Miller, and David J.
Lipman (1997), ″Gapped BLAST and PSI-BLAST: a new generation of
protein database search programs″, Nucleic Acids Res. 25: 3389-3402.
Database: Triticum aestivum.IWGSC.dna.toplevel
22 sequences; 14, 547, 261, 565 total letters
Query = TaFad_e6_R
Length = 21

| | |
|---|---|
| | Score |
| E | (Bits) |
| Sequences producing significant alignments: | |
| Value | |
| EG:6D dna:chromosome chromosome:IWGSC:6D:1:473592718:1 REF | 42.1 |
| 0.009 | |
| EG:6B dna:chromosome chromosome:IWGSC:6B:1:720988478:1 REF | 42.1 |
| 0.009 | |
| EG:6A dna:chromosome chromosome:IWGSC:6A:1:618079260:1 REF | 0.009 |
| 42.1 | |

>EG:6D dna:chromosome chromosome:IWGSC:6D:1:473592718:1 REF
Length = 473592718
Score = 42.1 bits (21), Expect = 0.009
Identities = 21/21 (100%), Gaps = 0/21 (0%)
Strand = Plus/Plus

| | | |
|---|---|---|
| Query 1 | TCCGTCAGCTCAGCTTTGGCA | 21 |

|||||||||||||||||||||

| | | |
|---|---|---|
| Sbjct 46855595 | TCCGTCAGCTCAGCTTTGGCA | 46855615 |

>EG:6B dna:chromosome chromosome:IWGSC:6B:1:720988478:1 REF
Length = 720988478
Score = 42.1 bits (21), Expect = 0.009
Identities = 21/21 (100%), Gaps = 0/21 (0%)
Strand = Plus/Plus

| | | |
|---|---|---|
| Query 1 | TCCGTCAGCTCAGCTTTGGCA | 21 |

|||||||||||||||||||||

| | | |
|---|---|---|
| Sbjct 115946328 | TCCGTCAGCTCAGCTTTGGCA | 115946348 |

>EG:6A dna:chromosome chromosome:IWGSC:6A:1:618079260:1 REF
Length = 618079260
Score = 42.1 bits (21), Expect = 0.009
Identities = 21/21 (100%), Gaps = 0/21 (0%)
Strand = Plus/Plus

| | | |
|---|---|---|
| Query 1 | TCCGTCAGCTCAGCTTTGGCA | 21 |

|||||||||||||||||||||

| | | |
|---|---|---|
| Sbjct 60443372 | TCCGTCAGCTCAGCTTTGGCA | 60443392 |

| | | |
|---|---|---|
| Lambda | KH | H |
| 1.37 | 0.711 | 1.31 |

Gapped

| | | |
|---|---|---|
| Lambda | KH | H |
| −1.00 | −1.00 | −1.00 |

Effective search space used: 43641783507
3. Results for Job 3: pinb_F
BLASTN 2.9.0+
Reference: Stephen F. Altschul, Thomas L. Madden, Alejandro A.
Schaffer, Jinghui Zhang, Zheng Zhang, Webb Miller, and David J.
Lipman (1997), ″Gapped BLAST and PSI-BLAST: a new generation of
protein database search programs″, Nucleic Acids Res. 25: 3389-3402.
Database: Triticum aestivum.IWGSC.dna.toplevel
22 sequences; 14, 547, 261, 565 total letters
Query = pinb_F
Length = 20

| | |
|---|---|
| | Score |
| E | (Bits) |
| Sequences producing significant alignments: | |
| Value | |
| EG:5D dna:chromosome chromosome:IWGSC:5D:1:566080677:1 REF | 40.1 |
| 0.024 | |

>EG:5D dna:chromosome chromosome:IWGSC:5D:1:566080677:1 REF
Length = 566080677
Score = 40.1 bits (20), Expect = 0.024
Identities = 20/20 (100%), Gaps = 0/20 (0%)
Strand = Plus/Plus

| | | |
|---|---|---|
| Query 1 | AGGCAGGCTCGGTGGCTTCT | 20 |

||||||||||||||||||||

| | | |
|---|---|---|
| Sbjct 3609989 | AGGCAGGCTCGGTGGCTTCT | 3610008 |

| | | |
|---|---|---|
| Lambda | K | H |
| 1.37 | 0.711 | 1.31 |

Gapped

| | | |
|---|---|---|
| Lambda | K | H |
| −1.00 | −1.00 | −1.00 |

Effective search space used: 29094522338
Database: Triticum aestivum.IWGSC.dna.toplevel
Posted date: Aug. 4, 2020 11:54 AM
Number of letters in database: 14,547,261, 565
Number of sequences in database: 22
Matrix: blastn matrix 1 −3
4. Results for Job 4: pinb-R
BLASTN 2.9.0+
Reference: Stephen F. Altschul, Thomas L. Madden, Alejandro A.
Schaffer, Jinghui Zhang, Zheng Zhang, Webb Miller, and David J.
Lipman (1997), ″Gapped BLAST and PSI-BLAST: a new generation of
protein database search programs″, Nucleic Acids Res. 25: 3389-3402.
Database: Triticum aestivum. IWGSC.dna. toplevel
22 sequences; 14, 547, 261, 565 total letters
Query = pinb-R
Length = 20

| | |
|---|---|
| | Score |
| E | (Bits) |
| Sequences producing significant alignments: | |
| Value | |
| EG:5D dna: chromosome chromosome:IWGSC:5D:1:566080677:1 REF | 40.1 |
| 0.024 | |

>EG:5D dna:chromosome chromosome:IWGSC:5D:1:566080677:1 REF
Length = 566080677
Score = 40.1 bits (20), Expect = 0.024
Identities = 20/20 (100%), Gaps = 0/20 (0%)
Strand = Plus/Minus

| | | |
|---|---|---|
| Query 1 | TCGGCGCCCATGTTGCACTT | 20 |

||||||||||||||||||||

| | | |
|---|---|---|
| Sbjct 3610087 | TCGGCGCCCATGTTGCACTT | 3610068 |

| | | |
|---|---|---|
| Lambda | K | H |
| 1.37 | 0.711 | 1.31 |

Gapped

| | | |
|---|---|---|
| Lambda | K | H |
| −1.00 | −1.00 | −1.00 |

Effective search space used: 29094522338
Database: Triticum aestivum.IWGSC.dna.toplevel
Posted date: Aug. 4, 2020 11:54 AM
Number of letters in database: 14,547,261, 565
Number of sequences in database: 22
Matrix: blastn matrix 1 -3
5. Results for Job 5: OAsense_i_F
BLASTN 2.9.0+
Reference: Stephen F. Altschul, Thomas L. Madden, Alejandro A.
Schaffer, Jinghui Zhang, Zheng Zhang, Webb Miller, and David J.
Lipman (1997), ″Gapped BLAST and PSI-BLAST: a new generation of
protein database search programs″, Nucleic Acids Res. 25: 3389-3402.
Database: Triticum aestivum.IWGSC.dna.toplevel
22 sequences; 14, 547, 261, 565 total letters
Query = OAsense i F
Length = 20

| | |
|---|---|
| E | Score |
| Sequences producing significant alignments: | (Bits) |
| Value | |
| EG:6B dna:chromosome chromosome:IWGSC:6B:1:720988478:1 REF | 40.1 |
| 0.024 | |

>EG:6B dna:chromosome chromosome:IWGSC:6B:1:720988478:1 REF
Length = 720988478
Score = 40.1 bits (20), Expect = 0.024
Identities = 20/20 (100%), Gaps = 0/20 (0%)
Strand = Plus/Plus

| | | |
|---|---|---|
| Query 1 | CGGTGCCAGGCCATCCACAA | 20 |

||||||||||||||||||||

| | | |
|---|---|---|
| Sbjct 43481368 | CGGTGCCAGGCCATCCACAA | 43481387 |

| | | |
|---|---|---|
| Lambda | K | H |
| 1.37 | 0.711 | 1.31 |

Gapped

| | | |
|---|---|---|
| Lambda | K | H |
| -1.00 | -1.00 | -1.00 |

Effective search space used: 29094522338
Database: Triticum aestivum.IWGSC.dna.toplevel
Posted date: Aug. 4, 2020 11:54 AM
Number of letters in database: 14, 547, 261, 565
Number of sequences in database: 22
Matrix: blastn matrix 1 -3

1. A nucleic acid construct consisting of a nucleotide sequence of SEQ ID NO: 1 or a variant thereof.

2. An expression vector consisting of the nucleic acid construct of claim 1.

3. An isolated cell transfected with the nucleic acid construct of claim 1 or the expression vector consisting of the nucleic acid construct.

4. A genetically modified plant wherein said plant comprises the transfected cell of claim 3.

5. The genetically modified plant of claim 4, wherein the nucleotide sequence is incorporated into the genome in a stable form.

6. The genetically modified plant of claim 4, wherein the plant belongs to the genus Triticum.

7. The genetically modified plant of claim 6, wherein the plant is Triticum aestivum or Triticum turgidum.

8. The genetically modified plant of claim 7, wherein the plant is a Bobwhite cultivar.

9. A seed derived from the genetically modified plant of claim 4, wherein the seed comprises the nucleotide sequence defined in SEQ ID NO: 1 or a variant thereof.

10. The pollen, propagule, progeny or part of the plant derived from the genetically modified plant of claim 4, wherein the pollen, propagule, progeny or part comprise the nucleotide sequence defined in SEQ ID NO: 1 or a variant thereof.

11. A wheat plant, plant part or progeny thereof comprising the genotype of the wheat event T258, wherein a representative sample of seed of said wheat event has been deposited with the NCIMB under accession no. NCIMB 43626.

12. A seed derived from the wheat plant of claim 11 or derived from the progeny of the wheat plant, wherein preferably the seed comprises SEQ ID NO: 1 or a variant thereof.

13. A wheat plant, plant thereof or progeny grown from the seed of claim 12.

14. A seed comprising the genotype of the wheat event T258, wherein a representative sample of seed of said wheat event has been deposited with NCIMB under accession no. NCIMB 43626.

15. A genetically altered wheat plant or part thereof grown from the seed of claim 14.

16. A genetically altered seed produced from the wheat plant of claim 15, wherein the seed comprises the wheat event T258.

17. A genetically altered wheat plant or part thereof grown from the seed of claim 16.

18. (canceled)

19. (canceled)

20. A food composition prepared from the seed of claim 9.

21. A method for obtaining the genetically modified plant of claim 4, comprising the following:
(a) selecting a part of the plant;
(b) transfecting at least one cell or the part of the plant of paragraph (a) with the nucleic acid construct consisting of a nucleotide sequence of SEQ ID NO: 1 or a variant thereof;
(c) regenerating at least one plant derived from the transfected cell or cells;
(d) selecting one or more plants obtained according to paragraph (c) that show silencing or reduced expression and/or content of at least one of alpha, beta, gamma, and omega gliadins, reduced total gliadin content, reduced gluten content, reduced immunoreactivity and/or increased expression and/or content of glutenins.

22. A method of reducing the expression and/or content of at least one of alpha, beta, gamma, and omega gliadins, reducing total gliadin content, reducing gluten content, reducing immunoreactivity and/or increasing expression and/or content of glutenins, the method comprising introducing and expressing in a plant the nucleic acid construct of claim 1.

23. A method for producing a food composition with reduced expression and/or content of at least one of alpha, beta, gamma, and omega gliadins, reduced total gliadin content, reduced gluten content, reduced immunoreactivity and/or increased expression and/or content of glutenins, the method comprising producing a plant or part thereof in which alpha, beta, gamma, and omega gliadins are silenced according to claim 17 and preparing a food composition from said seeds.

24. The method of claim 21, wherein the plant belongs to the genus Triticum.

25. The method of claim 24, wherein the plant is Triticum aestivum.

26. A method for modulating an immune response to gliadins and/or gluten, the method comprising providing a diet of a food composition produced according to claim 20.

27. A wheat plant having at least one chromosome having a transgene/genomic junction polypeptide of SEQ ID NO: 28 or SEQ ID NO: 29.

28. (canceled)

29. (canceled)

30. A pair of DNA molecules comprising a first DNA molecule as defined in SEQ ID NO: 20 or 21 or a complementary sequence or variant thereof and a second DNA molecule as defined in SEQ ID NO: 26 or 27 or a complementary sequence or a variant thereof, wherein said sequences can function as primers or probes for detecting transgenic event T258 in a wheat tissue sample.

31. A kit for the identifying transgenic event T258 in a wheat tissue sample, wherein the kit comprises at least one probe that selectively detects SEQ ID NO: 1 in a specific region or the complement of SEQ ID NO: 1, wherein the probe comprises a pair of DNA molecules as defined in SEQ ID NO: 30.

32. A method of producing a wheat plant with reduced expression and/or content of at least one of alpha, beta, gamma, and omega gliadins, reduced total gliadin content, reduced gluten content, reduced immunoreactivity and/or increased expression and/or content of glutenins, the method comprising:
(a) sexually crossing a first parent wheat plant, where the first parent wheat plant comprises the wheat event T258 with the genotype deposited at the NCIMB under accession no. NCIMB 43626, with a second wheat plant, whereby the second wheat plant does not comprise the wheat event T258, to produce a first generation of progeny plants;
(b) selecting plants from the first progeny with reduced gliadin and/or gluten content;
(c) crossing plants obtained in step b) with each other to produce a second generation of progeny plants; and
(d) selecting the from the second-generation progeny plants, plants with reduced gliadin and/or gluten content as described herein.

33. The method of claim 32, wherein the method further comprises backcrossing the second generation progeny plants with the second parent to produce a plurality of backcross progeny plants.


SEQUENCE LISTING (33 sequences)

SEQ ID NO: 1 - DNA, 3577 bp, Triticum aestivum
gcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcttccagaaaaaactttgctaatgtatgacagttatgtagtgaatattttcaacctaaggaacatttttaatttattttttataaaattataattcgacttggcattcgaatttggatttgagttttggtttgaaacggaaagaggattagtaaaatgattatgatgacatagcatcattaggcatgagattactgtagcatgacatgggggtgttacacttgtacaatattcctacccttgacataaaaggagaatttgatgagtcatgtattgataacgtatacaacattactacccttgacataaaaggagaatttgatgagtcatgcattgataacatgtacaagattactatcagcttgttcatcttaccatcatattatacaacactacaagttagttttagaaagaacaagagtccacaacaaatatcagaatacttgcctgatctatcttaacaacatgcacaaggacacaaatttagtcccccgcaagctatgaagatttggtttatgtctaacaacttgtacagatccaaaaggaatgcaatccagataattgtttgacatgtaaagtgaataagatgagtcaatgccaattatcaagtattcctcactcttagatgatatgtacaataaaaagacaactttgatgatcactctgaaattacgtttgtatgtagtgccaccaaacacaacataccaaataattagtttgataagcatcaaatcacttttaaaaaagaaagcaataatgaaaagaaacctaaccatggtagcyataaaaaggcctacaatatgtagactccataccatcatccatcgttcacacaactagagcacaagcagaaaatcaaagtacgtagtagttaacgcaaatccaccctcgaggtcatcaccactttgtacaagaaagctgggtcgaattcgcccttccttcctcatctttgtcctccttgccatggcgatgaagatcgccactgccgctagggagttaaaccctagcaacaaagagttacaatcacctcaacaatcattttcccatcaacaacaaccatttccacagcagccatatccacaacaaccatatccatcacagcaaccatatccatcgcaacaaccatttcaacaacaactgattccatgcagggatgttgtattgcaacaacacagcatagcgtatggaagctcacaagttttgcaacaaagtacttaccagctggtgcaacaattgtgttgtcagcagctgtggcagatccccgagcagtcgcggtgccaggccatccacaatgttataagggcgaattcggagcctgcttttttgtacaaacttgtgatgacggtatcgataagcttgatatccacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttgatatcctgcagattcgcccttatcacaagtttgtacaaaaaagcaggctccgaattcgcccttataacattgtggatggcctggcaccgcgactgctcggggatctgccacagctgctgacaacacaattgttgcaccagctggtaagtactttgttgcaaaacttgtgagcttccatacgctatgctgtgttgttgcaatacaacatccctgcatggaatcagttgttgttgaaatggttgttgcgatggatatggttgctgtgatggatatggttgttgtggatatggctgctgtggaaatggttgttgttgatgggaaaatgattgttgaggtgattgtaactctttgttgctagggtttaactccctagcggcagtggcgatcttcatcgccatggcaaggaggacaaagatgaggaaggaagggcgaattcgacccagctttcttgtacaaagtggtgtaagggcgaattccagcacactggcggccgttactagtggatccgagctcatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgatgataagctgtcgaacagaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc

SEQ ID NO: 2 - DNA, 30 bp, Artificial Sequence
  Overlapping Omega III_R primer
cagttgttgttgaaatggttgttgcgatgg

SEQ ID NO: 3 - DNA, 23 bp, Artificial Sequence
  prGli_F primer
ttccagaaaaaactttgctaatg

SEQ ID NO: 4 - DNA, 20 bp, Artificial Sequence
  OAsense_i-F
cggtgccaggccatccacaa

SEQ ID NO: 5 - DNA, 20 bp, Artificial Sequence
  OAsense_i-R
gcggcgtaccttgaagcgga

SEQ ID NO: 6 - DNA, 22 bp, Artificial Sequence
  TaFAD6-F
gcttggcattcggaaggaggat

SEQ ID NO: 7 - DNA, 21 bp, Artificial Sequence
  TaFAD6-R
tccgtcagctcagctttggca

SEQ ID NO: 8 - DNA, 20 bp, Artificial Sequence
  pinb-F
aggcaggctcggtggcttct

SEQ ID NO: 9 - DNA, 20 bp, Artificial Sequence
  pinb-R
tcggcgcccatgttgcactt

SEQ ID NO: 10 - DNA, 48 bp, Artificial Sequence
  Genome Walker Adapter
gtaatacgactcactatagggcacgcgtggtcgacggcccgggctggt

SEQ ID NO: 11 - DNA, 28 bp, Artificial Sequence
  Primer - GW3T258-GSP1 Downstream NOS terminator
gcataaagtgtaaagcctggggtgccta

SEQ ID NO: 12 - DNA, 27 bp, Artificial Sequence
  Primer - GW3T258-GSP2 Downstream NOS terminator
gccagctgcattaatgaatcggccaac

SEQ ID NO: 13 - DNA, 28 bp, Artificial Sequence
  Primer - GW5T258-GSP1 Upstream Gamma-gliadin promoter
tttacaacgtcgtgactgggaaaaccct

SEQ ID NO: 14 - DNA, 28 bp, Artificial Sequence
  Primer - GW5T258-GSP2 Upstream Gamma-gliadin promoter
tacccaacttaatcgccttgcagcacat

SEQ ID NO: 15 - DNA, 22 bp, Artificial Sequence
  Primer - AP1 Genome Walker Adaptor
gtaatacgactcactatagggc

SEQ ID NO: 16 - DNA, 19 bp, Artificial Sequence
  Primer - AP2 Genome Walker Adaptor
actatagggcacgcgtggt

SEQ ID NO: 17 - DNA, 845 bp, Triticum aestivum
tgcaagagatcgagcgcgctctccgcacggtacgacgatgcgtgccggaaggaagtgcggtgcatcttcccaatggtgcagccctcgaagaactggtcgaggtgcgcgattgctggcatgccgcgcaccatgccctttgcaccgaggtcatgcagcgcgcggaaatgcagatgcccgaatcttgcatgccacctccaggcgtcgtcctgaactccggcgaggaggcacacgggtatggccaagttcagagccacggcgtaaagccggttgctcgtccgccgcacccgcgcgagtagcaacctctgcctgtcatgcaggcagagttccccatcctcgatcacggctttgcagccgtgctcatcgagctgcccgacgctgacgatgcttgtgcgtagctgctggatgtagtagacgttggcgagcgcgcgatgttcaccgttgcggcacgtgaagagcacgacgccgcacccacaaatctgcacgaccgagccgtcaccaaaacatacagtacctcgtaccgtctcgtcgagctccgagagcatgtcgcggcgccctgtcatgtgattgctagcgccggtgtcgaggtaccaccggtcgtcgtccgtcgccacgggcacgacccgtgcttcgttgaggaagatctccatgcagtctgtttcgcaatctgtgtcaattggcccagctatcctgtgggctgctgctctatgggccgaatccgaggcctctggttaattgggccgtcggccagcccattccagcgcatcgctaaaagatgtcgccattcgccattcaggctgcgcaactgttgggaagggcgatcggtgcgggcctcttcgctatacgcc

SEQ ID NO: 18 - DNA, 355 bp, Triticum aestivum
tgccagctgcattaatgaatcggccaacgcgcggggagctcctagtcggcgccttaagcgccggctaggagaagcagcgcccacgcgccctccaagtgggccggcccatgaaacgcgtcgtacgcgaaaacccaccaaaaaagaggatactacgggattcgaactaaggatgttcactccacgtgcaaagcaagctgaccacttgagctaccgaccgagagtgattacaaacagcgtgacactgtaaataacattgtcgcggcgctactgttgcacataacaatgtactatacatcgcatcaaatatttgggaacaacaaaaatcgtgactagaaaaggttcatggatttgaaaa

SEQ ID NO: 19 - DNA, 19 bp, Artificial Sequence
  Primer - oSS333
actggcacttgctgatgct

SEQ ID NO: 20 - DNA, 19 bp, Artificial Sequence
  Primer - oSS334
gtgcagccctcgaagaact

SEQ ID NO: 21 - DNA, 19 bp, Artificial Sequence
  Primer - oSS337
gcgccgcgacaatgttatt

SEQ ID NO: 22 - DNA, 19 bp, Artificial Sequence
  Primer - oSS338
cccttaaaggccaacccgt

SEQ ID NO: 23 - DNA, 19 bp, Artificial Sequence
  Primer - oSS339
catcgatggctcaggcgat

SEQ ID NO: 24 - DNA, 19 bp, Artificial Sequence
  Primer - pDhpA/BZR-F1
ccagtgccaagcttgcatg

SEQ ID NO: 25 - DNA, 19 bp, Artificial Sequence
  Primer - pDhpA/BZR-R1
gcaccccaggctttacact

SEQ ID NO: 26 - DNA, 19 bp, Artificial Sequence
  Primer - pDhpA/BZR-R2
aacacccccatgtcatgct

SEQ ID NO: 27 - DNA, 19 bp, Artificial Sequence
  Primer - pA/BZR-NOS-F2
cgcgcggtgtcatctatgt

SEQ ID NO: 28 - DNA, 1124 bp, Triticum aestivum
cccatttcatttacttttttctgtcagcaaaaagttcgctggagaggccaacccaggacttcctttgtttagcgtgaatgcaatgaccaaccagccaccctcacctagtgtgacgggttccatctttttttctattgtcttctttcttcttttttcttctgtacgttttctttcgtcttttcgttttttttctgtaccttttctcaaagaatgcgtgaatttgttttttgtaatatccgttgaacattttgcgatatccgttgacattttcttgaaataagctgaatattttaaataatacatggggaacttattttaatatacgatgaacattttttaaaaaataggaatatatgcgctgaagaatttttggacacgtactgaacattttgtgaaaatatgttgaacaatttgtgaatacacattgaacattgtgtaatatatactgaacaaaattaagaaacagagtgaacaattgtatactcattgaataaccaaaaccaaaatagaaaaaaaaaatcaaaaaatatgtatatatatatcctgaacatttttatatccattgaacattttaaataatacacgatgaacattttcaaaatccatgaaccttttctagtcacgatttttgttgttcccaaatatttgatgcgatgtatagtacattgttatgtgcaacagtagcgccgcgacaatgttatttacagtgtcacgctgtttgtaatcactctcggtcggtagctcaagtggtcagcttgctttgcacgtggagtgaacatccttagttcgaatcccgtagtatcctcttttttggtgggttttcgcgtacgacgcgtttcatgggccggcccacttggagggcgcgtgggcgctgcttctcctagccggcgcttaaggcgccgactaggagctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattctg

SEQ ID NO: 29 - DNA, 1871 bp, Triticum aestivum
atcataatcattttactaatcctctttccgtttcaaaccaaaactcaaatccaaattcgaatgccaagtcgaattataattttataaaaaataaattaaaaatgttccttaggttgaaaatattcactacataactgtcatacattagcaaagttttttctggaagcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgacatcgtttagcgatgcgctggaatgggctggccgacggcccagttaaccagaggcctcggattcggcccatagagcagcagcccacaggatagctgggccaattgacacagattgcgaaacagactgcatggagatcttcctcaacgaagcacgggtcgtgcccgtggcgacggacgacgaccggtggtacctcgacaccggcgctagcaatcacatgacagggcgccgcgacatgctctcggagctcgacgagacggtacgaggtactgtatgttttggtgacggctcggtcgtgcagatttgtgggtgcggcgtcgtgctcttcacgtgccgcaacggtgaacatcgcgcgctcgccaacgtctactacatccagcagctacgcacaagcatcgtcagcgtcgggcagctcgatgagcacggctgcaaagccgtgatcgaggatggggaactctgcctgcatgacaggcagaggttgctactcgcgcgggtgcggcggacgagcaaccggctttacgccgtggctctgaacttggccatacccgtgtgcctcctcgccggagttcaggacgacgcctggaggtggcatgcaagattcgggcatctgcatttccgccgctgcatgacctcggtgcaaagggcatggtgcgcggcatgccagcaatcgcgcacctcgaccagttcttcgagggctgcaccattgggaagatgcaccgcacttccttccggcacgcatcgtcgtaccgtgcggagagcgcgctcgagctcgtgcactggcgacttgtgtggtccgatcactccggcgacggccagcggtaacaagtattttctattgatagttgacgatttcagcagatacatgtggcttgaggtactgaaaagcaaggatgaggcatttcggtacttcaagaagatcaaggcgcacgcggacatacaaggaaacgccaagctccgggcgttccacacagacagaggtggcgagttcacctcgaacgaattcgccgcctactgtgaagaactgggcatccagaggaacacgacgacgccgtactctcctcaacaaaatggagtggtcgaacggagaaaccagattgtcggggagatggcccacagtctcatgaaggccatgggcgtgccgtgcgcgttctggggcgaggccgtgcgcaccgccgtccacatcctcaatcggtctccaaccagaagcctgcagggcatcacgccatacgagacgtggcgcaagaagaagccagcggtggactacttccgcaccttcggctgcgtcgtgcatgtcaagctcatcggaccaagagtgacaaagctggcggacagatcgcgtcctggcatcttcccggggtacgagctggggacgaagggatatcaggtgtacgacccgattgacaagcgtctgtacatcacacatgatgtgcgcttcgaggaggatcgccgctgggactggagcaaggacgccggcgactcgatgcacacctgtgagttcactgtcgtgtactccgacgcgggttcgcccgtaac

SEQ ID NO: 30 - DNA, 2468 bp, Triticum aestivum
cccatttcatttacttttttctgtcagcaaaaagttcgctggagaggccaacccaggacttcctttgtttagcgtgaatgcaatgaccaaccagccaccctcacctagtgtgacgggttccatctttttttctattgtcttctttcttcttttttcttctgtacgttttctttcgtcttttcgttttttttctgtaccttttctcaaagaatgcgtgaatttgttttttgtaatatccgttgaacattttgcgatatccgttgacattttcttgaaataagctgaatattttaaataatacatggggaacttattttaatatacgatgaacattttttaaaaaataggaatatatgcgctgaagaatttttggacacgtactgaacattttgtgaaaatatgttgaacaatttgtgaatacacattgaacattgtgtaatatatactgaacaaaattaagaaacagagtgaacaattgtatactcattgaataaccaaaaccaaaatagaaaaaaaaatcaaaaaatatgtatatatatatcctgaacatttttatatccattgaacattttaaataatacacgatgaacattttcaaaatccatgaaccttttctagtcacgatttttgttgttcccaaatatttgatgcgatgtatagtacattgttatgtgcaacagtagcgccgcgacaatgttatttacagtgtcacgctgtttgtaatcactctcggtcggtagctcaagtggtcagcttgctttgcacgtggagtgaacatccttagttcgaatcccgtagtatcctcttttttggtgggttttcgcgtacgacgcgtttcatgggccggcccacttggagggcgcgtgggcgctgcttctcctagccggcgcttaaggcgccgactaggagctccccgggactagatggatgatgtcgacatcgtttagcgatgcgctggaatgggctggccgacggcccagttaaccagaggcctcggattcggcccatagagcagcagcccacaggatagctgggccaattgacacagattgcgaaacagactgcatggagatcttcctcaacgaagcacgggtcgtgcccgtggcgacggacgacgaccggtggtacctcgacaccggcgctagcaatcacatgacagggcgccgcgacatgctctcggagctcgacgagacggtacgaggtactgtatgttttggtgacggctcggtcgtgcagatttgtgggtgcggcgtcgtgctcttcacgtgccgcaacggtgaacatcgcgcgctcgccaacgtctactacatccagcagctacgcacaagcatcgtcagcgtcgggcagctcgatgagcacggctgcaaagccgtgatcgaggatggggaactctgcctgcatgacaggcagaggttgctactcgcgcgggtgcggcggacgagcaaccggctttacgccgtggctctgaacttggccatacccgtgtgcctcctcgccggagttcaggacgacgcctggaggtggcatgcaagattcgggcatctgcatttccgcgcgctgcatgacctcggtgcaaagggcatggtgcgcggcatgccagcaatcgcgcacctcgaccagttcttcgagggctgcaccattgggaagatgcaccgcacttccttccggcacgcatcgtcgtaccgtgcggagagcgcgctcgakctcktgcacggcgacttgtgtggtccgatcactccggcgacggccagcggtaacaagtattttctattgatagttgacgatttcagcagatacatgtggcttgaggtactgaaaagcaaggatgaggcatttcggtacttcaagaagatcaaggcgcacgcggacatacaaggaaacgccaagctccgggcgttccacacagacagaggtggcgagttcacctcgaacgaattcgccgcctactgtgaagaactgggcatccagaggaacacgacgacgccgtactctcctcaacaaaatggagtggtcgaacggagaaaccagattgtcggggagatggcccacagtctcatgaaggccatgggcgtgccgtgcgcgttctggggcgaggccgtgcgcaccgccgtccacatcctcaatcggtctccaaccagaagcctgcagggcatcacgccatacgagacgtggcgcaagaagaagccagcggtggactacttccgcaccttcggctgcgtcgtgcatgtcaagctcatcggaccaagagtgacaaagctggcggacagatcgcgtcctggcatcttcccggggtacgagctggggacgaagggatatcaggtgtacgacccgattgacaagcgtctgtacatcacacatgatgtgcgcttcgaggaggatcgccgctgggactggagcaaggacgccggcgactcgatgcacacctgtgagttcactgtcgtgtactccgacgcgggttcgcccgtaac

SEQ ID NO: 31 - protein, 12 aa, Triticum aestivum
QPFPQPEQPFPW

SEQ ID NO: 32 - DNA, 24 bp, Triticum aestivum
aggcggtttgcgtattgggcgcgc

SEQ ID NO: 33 - DNA, 2691 bp, Triticum aestivum
cccttaaaggccaacccgtacagggcgcacacccccctctccccgctgggccggcccatttcatttacttttttctgtcagcaaaaagttcgctggagaggccaacccaggacttcctttgtttagcgtgaatgcaatgaccaaccagccaccctcacctagtgtgacgggttccatctttttttctattgtcttctttcttcttttttcttctgtacgttttctttcgtcttttcgttttttttctgtaccttttctcaaagaatgcgtgaatttgttttttgtaatatccgttgaacattttgcgatatccgttgacattttcttgaaataagctgaatattttaaataatacatggggaacttattttaatatacgatgaacattttttaaaaaataggaatatatgcgctgaagaatttttggacacgtactgaacattttgtgaaaatatgttgaacaatttgtgaatacacattgaacattgtgtaatatatactgaacaaaattaagaaacagagtgaacaattgtatactcattgaataaccaaaaccaaaatagaaaaaaaaatcaaaaaatatgtatatatatatcctgaacatttttatatccattgaacattttaaataatacacgatgaacattttcaaaatccatgaaccttttctagtcacgatttttgttgttcccaaatatttgatgcgatgtatagtacattgttatgtgcaacagtagcgccgcgacaatgttatttacagtgtcacgctgtttgtaatcactctcggtcggtagctcaagtggtcagcttgctttgcacgtggagtgaacatccttagttcgaatcccgtagtatcctcttttttggtgggttttcgcgtacgacgcgtttcatgggccggcccacttggagggcgcgtgggcgctgcttctcctagccggcgcttaaggcgccgactaggagctccccgggactagatggatgatgtcgacatcgtttagcgatgcgctggaatgggctggccgacggcccagttaaccagaggcctcggattcggcccatagagcagcagcccacaggatagctgggccaattgacacagattgcgaaacagactgcatggagatcttcctcaacgaagcacgggtcgtgcccgtggcgacggacgacgaccggtggtacctcgacaccggcgctagcaatcacatgacagggcgccgcgacatgctctcggagctcgacgagacggtacgaggtactgtatgttttggtgacggctcggtcgtgcagatttgtgggtgcggcgtcgtgctcttcacgtgccgcaacggtgaacatcgcgcgctcgccaacgtctactacatccagcagctacgcacaagcatcgtcagcgtcgggcagctcgatgagcacggctgcaaagccgtgatcgaggatggggaactctgcctgcatgacaggcagaggttgctactcgcgcgggtgcggcggacgagcaaccggctttacgccgtggctctgaacttggccatacccgtgtgcctcctcgccggagttcaggacgacgcctggaggtggcatgcaagattcgggcatctgcatttccgcgcgctgcatgacctcggtgcaaagggcatggtgcgcggcatgccagcaatcgcgcacctcgaccagttcttcgagggctgcaccattgggaagatgcaccgcacttccttccggcacgcatcgtcgtaccgtgcggagagcgcgctcgakctcktgcacggcgacttgtgtggtccgatcactccggcgacggccagcggtaacaagtattttctattgatagttgacgatttcagcagatacatgtggcttgaggtactgaaaagcaaggatgaggcatttcggtacttcaagaagatcaaggcgcacgcggacatacaaggaaacgccaagctccgggcgttccacacagacagaggtggcgagttcacctcgaacgaattcgccgcctactgtgaagaactgggcatccagaggaacacgacgacgccgtactctcctcaacaaaatggagtggtcgaacggagaaaccagattgtcggggagatggcccacagtctcatgaaggccatgggcgtgccgtgcgcgttctggggcgaggccgtgcgcaccgccgtccacatcctcaatcggtctccaaccagaagcctgcagggcatcacgccatacgagacgtggcgcaagaagaagccagcggtggactacttccgcaccttcggctgcgtcgtgcatgtcaagctcatcggaccaagagtgacaaagctggcggacagatcgcgtcctggcatcttcccggggtacgagctggggacgaagggatatcaggtgtacgacccgattgacaagcgtctgtacatcacacatgatgtgcgcttcgaggaggatcgccgctgggactggagcaaggacgccggcgactcgatgcacacctgtgagttcactgtcgtgtactccgacgcgggttcgcccgtaaccacgacctacactgtgtcacccgaggtggtgggatcaccgcgagcgcgtgcaacgccaactacgccaagcacaccagttccactgcaatcccctcggacgcccgtgtccggcgggtcgggcgtgggcacgtcgagtgcgtcgagcgtgccagcatcagcaagtgccagt